(19) United States
(12) Patent Application Publication
(10) Pub. No.: US 2013/0134332 A1
(43) Pub. Date: May 30, 2013

(54) SHIELD AND METHOD FOR USING SAME

(71) Applicant: Leslie Ciancibello, Mentor, OH (US)

(72) Inventor: Leslie Ciancibello, Mentor, OH (US)

(73) Assignee: UNIVERSITY HOSPITALS OF CLEVELAND, Cleveland, OH (US)

(21) Appl. No.: 12/762,641

(22) Filed: Apr. 19, 2010

Related U.S. Application Data
(60) Provisional application No. 61/212,942, filed on Apr. 17, 2009.

(51) Int. Cl. G21F 1/08; A61B 6/03

(52) U.S. Cl. 250/519.1; 378/4

(57) ABSTRACT

A radiation shield for use in connection with computed tomography (CT) calcium scoring of a target area of a patient is provided. The shield is configured for placement over the target area and over one or more radiosensitive secondary areas adjacent to the target area such that x-rays are transmitted through the radiation shield and the target area for detection by an x-ray detector. The detected radiation can be processed for use in a calcium scoring procedure. The radiation shield is configured to reduce radiation exposure of the radiosensitive secondary areas during the calcium scoring procedure without substantially reducing the accuracy of the calcium score associated with the target area.

### RELATED APPLICATION DATA

The present application claims the benefit of U.S. Provisional Application Ser. No. 61/212,942, filed Apr. 17, 2009, the disclosure of which is herein incorporated by reference in its entirety.

### FIELD OF THE INVENTION

The present invention relates generally to a shield, and more particularly, to a radiation shield for use in diagnostic or therapeutic procedures as well as methods for the use of such shields.

### BACKGROUND OF THE INVENTION

Imaging systems include a source that emits signals (including but not limited to x-ray, radio frequency, or sonar signals), and the signals are directed toward an object to be imaged. The emitted signals and the interposed object interact to produce a response that is received by one or more detectors. The imaging system then processes the detected response signals to generate an image of the object.

For example, in computed tomography (CT) imaging, an x-ray source projects a fan-shaped beam which is collimated to lie within an X-Y plane of a Cartesian coordinate system and generally referred to as an “imaging plane”. The x-ray beam passes through an object being imaged, such as a patient. The beam, after being attenuated by the object, impinges upon an array of radiation detectors. The intensity of the attenuated radiation beam received at the detector array is dependent upon the attenuation of an x-ray beam by the object. Each detector element of the array produces a separate electrical signal that is a measurement of the beam intensity at the detector location. The intensity measurements from all the detectors are acquired separately to produce a transmission profile.

In third generation CT systems, the x-ray source and the detector array are rotated with a gantry within the imaging plane and around the object to be imaged such that the angle at which the x-ray beam intersects the object constantly changes. A group of x-ray attenuation measurements, i.e., projection data, from the detector array at one gantry angle is referred to as a “view”. A “scan” of the object comprises a set of views made at different gantry angles, or view angles, during one revolution of the x-ray source and detector.

In an axial scan, the projection data is processed to construct an image that corresponds to a two-dimensional slice taken through the object. One method for reconstructing an image from a set of projection data is referred to in the art as the filtered back projection technique. This process converts the attenuation measurements from a scan into integers called “CT numbers” or “Hounsfield units” (HU), which are used to control the brightness of a corresponding pixel on, for example, a cathode ray tube display.

It is known to use imaging data to identify evidence of disease by detecting and quantifying, i.e. “scoring”, substances that may be present in a patient's system. For example, CT images of the heart may be analyzed to quantify amounts of calcium in coronary regions of interest. Scoring is based upon the volume and Hounsfield unit of a calcified region. A number called the “calcium score” expresses the quantity of calcium present in the patient's coronary arterial system and may be used to assess coronary heart disease (CAD) risk and to study the progression of atherosclerotic plaque in the coronary arteries. Such CT images of the heart during calcium scoring procedures are taken without shielding the patient's breasts.

The importance of accurately identifying a patient's calcium score has increased as heart disease is one of the primary causes of mortality of men in the United States and is growing in prevalence in the rest of the world. In addition, increased public sensitivity of under diagnosed heart disease in women has resulted in an increased use of calcium scoring in the female population. However, women are often concerned about radiation exposure to their breasts due to higher occurrences of breast cancer. Therefore, while early recognition of heart disease is valuable, the amount of radiation exposure during CT calcium scoring procedures has been of increasing public concern. This may result in patients, including those with high risk of Coronary Artery Disease (CAD), to avoid calcium scoring procedures altogether.

To reduce the total scan time (and thereby the patient's radiation exposure), a “helical” scan may be performed. To perform a “helical” scan, the patient is moved while the data for the prescribed number of slices is acquired. Such a system generates a single helix from a fan beam helical scan. The helix mapped out by the fan beam yields projection data from which images in each prescribed slice may be reconstructed.

Reconstruction algorithms for helical scanning typically use helical weighing algorithms that weight the collected data as a function of view angle and detector channel index. Specifically, prior to a filtered back projection process, the data is weighted according to a helical weighing factor, which is a function of both the gantry angle and detector angle. The weighted data is then processed to generate CT numbers and to construct an image that corresponds to a two-dimensional slice taken through the object.

To further reduce the total acquisition time, multi-slice CT has been introduced. In multi-slice CT, multiple rows of projection data are acquired simultaneously at any time instant. When combined with helical scan mode, the system generates a single helix of cone beam projection data. Similar to the single slice, helical weighting scheme, a method can be derived to multiply the weight with the projection data prior to the filtered back projection algorithm. In some CT imaging systems, imaging is synchronized with an electrocardiogram (ECG or EKG) signal monitoring the patient, so that images representing the same phase of the patient's cardiac cycle can be taken during different cardiac cycles. For example, the CT imaging system may use an ECG signal to adapt triggering times for imaging to center the acquired phases at end systole and end diastole.

By employing a dual energy scanning protocol in which two scans are taken at a different peak energy, material discrimination in CT imaging can be used to improve differentiation of materials having similar CT number but different material attenuation properties, for example, calcium and iodine. This technique can be used with an appropriate acquisition method to perform a single CT cardiac examination within a breath hold that can take the place of two standard CT exams, one with contrast and one without contrast. A dual energy cardiac protocol can produce more robust calcium scoring and better vessel characterization in cases in which calcified plaque is present in iodinate coronary arteries. However, such dual kVp techniques may also result in an increased radiation dose to a patient, relative to some other known diagnostic techniques.

### SUMMARY OF THE INVENTION

The present invention provides a radiation shield for use in connection with calcium scoring procedures and an associated calcium scoring method in which the radiation shield is configured for placement over a target area as well as one or more adjacent radiosensitive secondary areas. The shield is configured for placement over the target area and over one or more radiosensitive secondary areas adjacent to the target area such that x-rays are transmitted through the radiation shield and the target area for detection by an x-ray detector. The detected radiation can be processed for use in a calcium scoring procedure. The radiation shield is configured to reduce radiation exposure of the radiosensitive secondary areas during the calcium scoring procedure without substantially reducing the accuracy of the calcium score associated with the target area.

One aspect of the disclosed technology relates to a method of generating a diagnostic output for a target area located adjacent a radiosensitive secondary area using a computed tomography (CT) device, the CT device having an x-ray source and an x-ray detector, that includes transmitting x-rays from the x-ray source through a radio-absorbent shield, the target area and the radiosensitive secondary area to the x-ray detector; receiving x-rays from the x-ray source at the x-ray detector; generating signals based on the received x-rays; and processing signals generated by the x-ray detector to provide a diagnostic output related to the target area.

According to another embodiment, the method includes positioning a patient between the x-ray source and the x-ray detector to scan a patient cross section including the target area and the radiosensitive secondary area; and placing the radio-absorbent shield over the radiosensitive secondary area and over the target area.

According to another embodiment, the target area includes coronary arteries.

According to another embodiment, the radiosensitive secondary area includes breast tissue.

According to another embodiment, the processing includes generating a calcium score representative of coronary artery calcification in the target area.

Another aspect of the disclosed technology relates to a method of generating a calcium score corresponding to a target area of a patient, wherein the target area is adjacent to a radiosensitive secondary area, that includes transmitting x-rays from an x-ray source through a shield positioned over the target area and the radiosensitive secondary area, through the target area and the radiosensitive secondary area to at least one x-ray detector, wherein the shield is radio-absorbent; receiving x-rays from the x-ray source at the at least one x-ray detector; generating signals based on the received x-rays; and processing signals generated by the at least one x-ray detector to generate a calcium score corresponding to the target area.

According to another embodiment, the method includes positioning a patient between the x-ray source and the x-ray detector to scan a patient cross section including the target area and the radiosensitive secondary area; and placing the shield over the radiosensitive secondary area and over the target area.

According to another embodiment, the target area includes coronary arteries.

According to another embodiment, the radiosensitive secondary area includes breast tissue.

According to another embodiment, the shield includes a plurality of layers of radio-absorbent material; a cover at least partially surrounding the plurality of layers of radio-absorbent material; wherein the shield is configured for placement over the target area and over a radiosensitive area adjacent to the target area; and wherein the plurality of layers of radio-absorbent material have a Pb equivalent of up to about 0.240 mm Pb equivalent.

According to another embodiment, the plurality of layers of radio-absorbent material have a Pb equivalent from about 0.060 mm Pb equivalent to about 0.240 mm Pb equivalent.

According to another embodiment, the shield includes four layers of radio-absorbent material, with each of the four layers of radio-absorbent material having a Pb equivalent of about 0.060 mm Pb equivalent.

According to another embodiment, the shield is configured for placement over a cardiac target area and the radiosensitive secondary area includes breast tissue adjacent to the cardiac area.

Another aspect of the disclosed technology relates to a shield for use in connection with computed tomography (CT) calcium scoring of a target area of a patient, that includes a plurality of layers of radio-absorbent material; a cover at least partially surrounding the plurality of layers of radio-absorbent material; wherein the shield is configured for placement over the target area and over a radiosensitive secondary area adjacent to the target area; and wherein the plurality of layers of radio-absorbent material have a Pb equivalent of up to about 0.240 mm Pb equivalent.

According to another embodiment, the plurality of layers of radio-absorbent material have a Pb equivalent from about 0.060 mm Pb equivalent to about 0.240 mm Pb equivalent.

According to another embodiment, the shield includes four layers of radio-absorbent material, with each of the four layers of radio-absorbent material having a Pb equivalent of about 0.060 mm Pb equivalent.

According to another embodiment, the shield is configured for placement over a cardiac target area and the radiosensitive area includes breast tissue adjacent to the cardiac area.

Another aspect of the disclosed technology relates to a method for detecting coronary artery calcification using a computed tomography (CT) system having an x-ray source and at least one x-ray detector that includes positioning a patient between the x-ray source and the at least one x-ray detector to scan a patient cross section including a target area and a radiosensitive secondary area; placing a shield partially transparent to x-rays over the radiosensitive secondary area and over the target area; transmitting x-rays from the x-ray source through the shield, the target area and the radiosensitive secondary area to the at least one x-ray detector; receiving x-rays from the x-ray source at the x-ray detector; generating signals based on the received x-rays; and processing signals generated by the at least one x-ray detector to generate a calcium score corresponding to the target area.

According to another embodiment, the shield includes a plurality of layers of radio-absorbent material; a cover at least partially surrounding the plurality of layers of radio-absorbent material; wherein the shield is configured for placement over the target area and over the radiosensitive secondary area adjacent to the target area; and wherein the plurality of layers of radio-absorbent material have a Pb equivalent of up to about 0.240 mm Pb equivalent.

According to another embodiment, the plurality of layers of radio-absorbent material have a Pb equivalent from about 0.060 mm Pb equivalent to about 0.240 mm Pb equivalent.

According to another embodiment, the shield includes four layers of radio-absorbent material, with each of the four layers of radio-absorbent material having a Pb equivalent of about 0.060 mm Pb equivalent.

Another aspect of the disclosed technology relates to a shield for reducing the x-ray dose of a radiosensitive area of a patient during computed tomography (CT) calcium scoring of the patient. The shield includes a body positionable over the radiosensitive area and a radio-absorbent material secured to the body that attenuates greater than 56% of the x-rays passing through the radio-absorbent material to the radiosensitive area without substantially affecting the accuracy of the calcium score.

According to another embodiment, the radio-absorbent material attenuates about 58% or more of the x-rays passing through the material to the radiosensitive area.

According to another embodiment, the radio-absorbent material attenuates about 60% or more of the x-rays passing through the material to the radiosensitive area.

According to another embodiment, the radio-absorbent material has a Pb equivalent of about 0.215 mm Pb or greater.

According to another embodiment, the radio-absorbent material has a Pb equivalent of about 0.22 mm Pb or greater.

According to another embodiment, the radio-absorbent material has a Pb equivalent of about 0.24 mm Pb or greater.

According to another embodiment, the x-rays attenuated by the radio-absorbent material are in the range of about 100 kV to about 120 kV.

According to another embodiment, the body is a polymer and the radio-absorbent material is impregnated therein.

According to another embodiment, the body is latex and the radio-absorbent material is bismuth.

These and further features of the present invention will be apparent with reference to the following description and attached drawings. In the description and drawings, particular embodiments of the invention have been disclosed in detail as being indicative of some of the ways in which the principles of the invention may be employed, but it is understood that the invention is not limited correspondingly in scope. Rather, the invention includes all changes, modifications and equivalents coming within the spirit and terms of the claims appended thereto.

Features that are described and/or illustrated with respect to one embodiment may be used in the same way or in a similar way in one or more other embodiments and/or in combination with or instead of the features of the other embodiments.

### BRIEF DESCRIPTION OF THE SEVERAL VIEWS OF THE DRAWINGS

Many aspects of the invention can be better understood with reference to the following drawings. The components in the drawings are not necessarily to scale, emphasis instead being placed upon clearly illustrating the principles of the present invention. Likewise, elements and features depicted in one drawing may be combined with elements and features depicted in additional drawings. Moreover, in the drawings, like reference numerals designate corresponding parts throughout the several views.

Objects and advantages, together with the operation of the invention, may be better understood by reference to the following detailed description taken in connection with the following illustrations, wherein:

FIG. 1 is a pictorial view of a CT imaging system.

FIG. 2 is a block schematic diagram of the system illustrated in FIG. 1.

FIG. 3 is a top view of one embodiment of a shield.

FIG. 4 is a cross-sectional view of the shield of FIG. 3.

FIG. 5 is a top view of illustrative examples of the shield.

FIG. 6 is a cross-sectional view of an anthropomorphic phantom and an ion chamber.

FIG. 7 is a cross-sectional view of a calibration insert for the anthropomorphic phantom of FIG. 6.

FIG. 8 is a cross-sectional view of an anthropomorphic phantom and an ion chamber covered by a shield.

FIG. 9 is a chart (Chart 1) showing the average radiation dosage (mR) vs. the number of layers of radioabsorbent material in a shield using a Siemens 16 Row: Siemens Somatom system.

FIG. 10 is a chart (Chart 2) showing the average radiation dosage (mR) vs. the number of layers of radioabsorbent material in a shield using a Philips 64: Philips Brilliance system.

FIG. 11 is a chart (Chart 3) showing the average total calcium score vs. the number of layers of radioabsorbent material in a shield using a Philips 64: Philips Brilliance system.

FIG. 12 is a chart (Chart 4) showing the average high density large calcium score vs. the number of layers of radioabsorbent material in a shield using a Philips 64: Philips Brilliance system.

FIG. 13 is a chart (Chart 5) showing the average low density small calcium score vs. the number of layers of radioabsorbent material in a shield using a Philips 64: Philips Brilliance system.

FIG. 14 is a chart (Chart 6) showing the average all density large calcium score vs. the number of layers of radioabsorbent material in a shield using a Philips 64: Philips Brilliance system.

FIG. 15 is a chart (Chart 7) showing the average all density small calcium score vs. the number of layers of radioabsorbent material in a shield using a Philips 64: Philips Brilliance system.

FIG. 16 is a chart (Chart 8) showing the average medium density large and small calcium score vs. the number of layers of radioabsorbent material in a shield using a Philips 64: Philips Brilliance system.

FIG. 17 is a chart (Chart 9) showing the average radiation dosage (mR) vs. the number of layers of radioabsorbent material in a shield using a Siemens 64 Row: Siemens Sensation system.

FIG. 18 is a chart (Chart 10) showing the average total calcium score vs. the number of layers of radioabsorbent material in a shield using a Siemens 64 Row: Siemens Sensation system.

FIG. 19 is a chart (Chart 11) showing the average high density large calcium score vs. the number of layers of radioabsorbent material in a shield using a Siemens 64 Row: Siemens Sensation system.

FIG. 20 is a chart (Chart 12) showing the average low density small calcium score vs. the number of layers of radioabsorbent material in a shield using a Siemens 64 Row: Siemens Sensation system.

FIG. 21 is a chart (Chart 13) showing the average all density large calcium score vs. the number of layers of radioabsorbent material in a shield using a Siemens 64 Row: Siemens Sensation system.

FIG. 22 is a chart (Chart 14) showing the average all density small calcium score vs. the number of layers of radioabsorbent material in a shield using a Siemens 64 Row: Siemens Sensation system.

FIG. 23 is a chart (Chart 15) showing the average medium density small and large calcium score vs. the number of layers of radioabsorbent material in a shield using a Siemens 64 Row: Siemens Sensation system.

FIG. 24 is a chart (Chart 16) showing the average radiation dosage (mR) vs. the number of layers of radioabsorbent material in a shield using a Siemens 64 Row: Siemens Dual Tube system.

FIG. 25 is a chart (Chart 17) showing the average total calcium score vs. the number of layers of radioabsorbent material in a shield using a Siemens 64 Row: Siemens Dual Tube system.

FIG. 26 is a chart (Chart 18) showing the average high density large calcium score vs. the number of layers of radioabsorbent material in a shield using a Siemens 64 Row: Siemens Dual Tube system.

FIG. 27 is a chart (Chart 19) showing the average low density small calcium score vs. the number of layers of radioabsorbent material in a shield using a Siemens 64 Row: Siemens Dual Tube system.

FIG. 28 is a chart (Chart 20) showing the average all density large calcium score vs. the number of layers of radioabsorbent material in a shield using a Siemens 64 Row: Siemens Dual Tube system.

FIG. 29 is a chart (Chart 21) showing the average all density small calcium score vs. the number of layers of radioabsorbent material in a shield using a Siemens 64 Row: Siemens Dual Tube system.

FIG. 30 is a chart (Chart 22) showing the average medium density large and small calcium score vs. the number of layers of radioabsorbent material in a shield using a Siemens 64 Row: Siemens Dual Tube system.

### DETAILED DESCRIPTION OF THE PREFERRED EMBODIMENTS

While the present invention is described with reference to embodiments described herein, it should be clear that the present invention is not limited to such embodiments. Therefore, the description of the embodiments herein is merely illustrative of the present invention and will not limit the scope of the invention.

As used herein, an element or step recited in the singular and proceeded with the word “a” or “an” should be understood as not excluding plural said elements or steps, unless such exclusion is explicitly recited. Furthermore, references to “one embodiment” of the present invention are not intended to be interpreted as excluding the existence of additional embodiments that also incorporate the recited features.

Also as used herein, the phrase “reconstructing an image” is not intended to exclude embodiments in which data representing an image is generated but a viewable image is not. However, many embodiments generate (or are configured to generate) at least one viewable image.

The present disclosure recognizes that a dual energy cardiac protocol can produce more robust calcium scoring and better vessel characterization in cases in which calcified plaque is present in iodinate coronary arteries. However, such dual kVp techniques may also result in an increased radiation dose to a patient, relative to some other known diagnostic techniques.

Although such efforts have been made to reduce a patient's radiation exposure without reducing the accuracy of the calcium score, it would be desirable to further reduce the level of radiation exposure to a patient during calcium scoring procedures.

Therefore, it would be desirable to provide a shield and a method of using the shield for reducing radiation exposure to the underlying tissue during calcium scoring procedures without substantially reducing the accuracy of the calcium score.

Technical effects of an embodiment of the present invention include, in various configurations, providing a shield over the tissue, such as the breast tissue, of a patient, acquiring data at particular times and at particular kVps, reconstruction of images using the acquired data, and/or determination of a calcium score for a lumen. These and other technical effects and the manner in which they are accomplished are described below.

A cardiac CT scan for coronary calcium obtains information about the presence, location and extent of calcified plaque in the coronary arteries. Calcified plaque is a build-up of fat and other substances, including calcium, and is a sign of CAD. Over time, the plaque buildup can narrow the arteries or close off blood flow to the heart. The result may be painful angina in the chest or a heart attack.

Because calcium is a marker of CAD, the amount of calcium detected on a cardiac CT scan is a helpful prognostic tool. The findings on cardiac CT are expressed as a calcium score that may be calculated using a variety of different factors known in the art including plaque volume and density. The type of treatment a patient will receive for CAD (if any) is typically dictated by the score. The extent of CAD is graded according to the calcium score: 0=No evidence of CAD; 1-10=Minimal evidence of CAD; 11-100=Mild evidence of CAD; 101-400=Moderate Evidence of CAD; >400=Extensive Evidence of CAD.

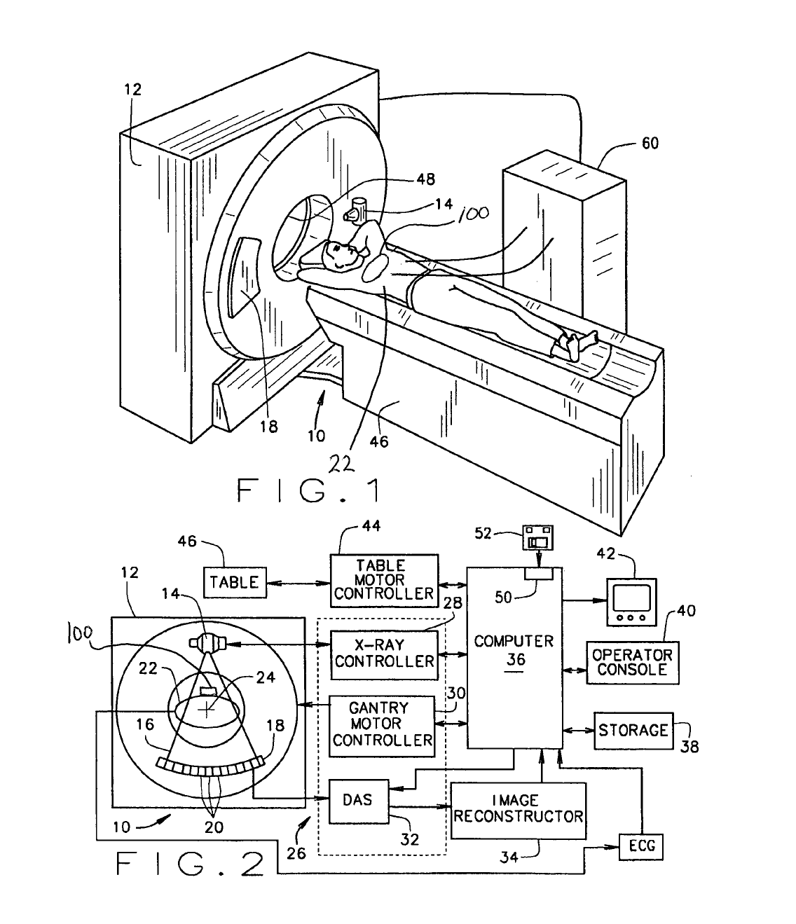

Referring to FIGS. 1 and 2, such calcium scoring may be assessed in a patient 22 with a multi-slice scanning imaging system, such as a CT imaging system 10. The CT imaging system 10 is shown as including a gantry 12 representative of a “third generation” CT imaging system. Gantry 12 has a radiation source such as x-ray tube 14 (also called x-ray source 14 herein) that projects a beam of x-rays 16 toward an x-ray detector, e.g., a detector array 18 on the opposite side of gantry 12. Detector array 18 may be formed by a plurality of detector rows (not shown) including a plurality of detector elements 20 which together sense the projected x-rays that pass through an object, such as a patient 22 between array 18 and source 14. Each detector element 20 produces an electrical signal that represents the intensity of an impinging x-ray beam and hence can be used to estimate the attenuation of the beam as it passes through the patient 22.

During a scan to acquire x-ray projection data, gantry 12 and the components mounted therein rotate about a center of rotation 24. Although FIG. 2 shows only a single row of detector elements 20 (i.e., a detector row), multi-slice detector array 18 may include a plurality of parallel detector rows of detector elements 20 such that projection data corresponding to a plurality of quasi-parallel or parallel slices can be acquired simultaneously during a scan.

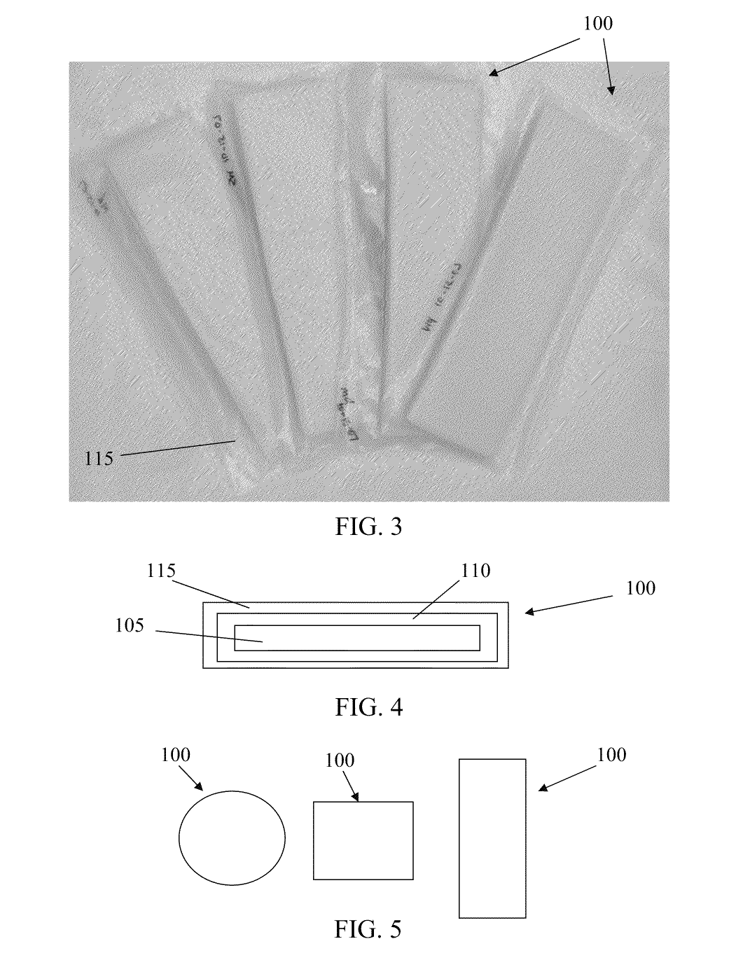

Such scans for calcium scoring are performed without shields, thereby exposing the breast tissue of the patient 22 to the full dosage of radiation emitted by the CT system. In an embodiment as best shown in FIG. 1, a shield 100 is provided that is capable of reducing the radiation exposure to the breast tissue of the patient 22 without reducing the accuracy of the calcium score. Although the shield 100 is shown as covering only a portion of the breast tissue of the patient 22, it is to be understood that the shield 100 may be sized and shaped to cover substantially all of the breast tissue. Although shown in FIG. 2 as substantially ovular in shape, it is to be understood that the shield 100 may be any shape, as shown in the illustrative examples in FIGS. 3, 4 and 5.

It is to be understood that the shield 100 may comprise a radioabsorbent material 105 including, but not limited to, bismuth, lead and mixtures thereof. In a nonlimiting example, the radioabsorbent material 105 may comprise a radioabsorbent material impregnated polymer. In an illustrative example, the radioabsorbent material impregnated polymer is a bismuth impregnated latex.

In a non-limiting example, the shield 100 may have a lead (“Pb”) equivalent of up to about 0.300 mm. In another non-limiting example, the shield 100 may have a Pb equivalent of greater than 0.040 mm Pb equivalent to about 0.300 mm Pb equivalent. In yet another non-limiting example, the shield 100 may have a Pb equivalent of from about 0.060 mm Pb equivalent to about 0.240 mm Pb equivalent.

In accordance with another non-limiting example, the shield 100 is configured to include a quantity (e.g., one or more layers of a given thickness and/or Pb equivalent) of a radio-absorbent material that attenuates greater than 56% of the x-rays passing through the radio-absorbent material without substantially affecting the accuracy of the calcium score. In accordance with another non-limiting example, the shield is configured to include a quantity (e.g., one or more layers of a given thickness and/or Pb equivalent) of radio-absorbent material that attenuates greater than 58% of the x-rays passing through the radio-absorbent material without substantially affecting the accuracy of the calcium score. In accordance with another non-limiting example, the shield is configured to include a quantity (e.g., one or more layers of a given thickness and/or Pb equivalent) of radio-absorbent material that attenuates greater than 60% of the x-rays passing through the radio-absorbent material without substantially affecting the accuracy of the calcium score. In accordance with one exemplary embodiment, the shield 100 includes a quantity of radio-absorbent material (e.g., one or more layers) having a Pb equivalent of about 0.215 mm Pb or greater. In accordance with another exemplary embodiment, the shield includes radio-absorbent material (e.g., one or more layers) having a Pb equivalent of about 0.22 mm Pb or greater. In accordance with another exemplary embodiment, the shield includes radio-absorbent material (e.g., one or more layers) having a Pb equivalent of about 0.24 mm Pb or greater.

As shown in FIGS. 3 and 4, the shield 100 may be provided with a cover 110. It is to be understood that the cover 110 may be any material including, but not limited to, foam. In a non-limiting example, the cover 110 may entirely surround the radioabsorbent material 105. In yet another illustrative example, a bag or seal 115 may be provided to enclose the radioabsorbent material 105 (and cover 110, if present) to prevent contamination and may be capable of being sanitized to allow the shield 100 to be reused.

As the x-ray tube 14 rotates to a point closest to the breast tissue (directly over the breast tissue of the patient 22) as best shown in FIG. 2, the shield 100 absorbs a portion of the beam of x-rays 16 without substantially reducing the accuracy of the CT image and resulting calcium score, thereby reducing the radiation exposure of the breast tissue of the patient 22. In a non-limiting example, the shield 100 reduces the radiation exposure of the breast tissue of the patient 22 by up to about 50%. In another non-limiting example, the shield 100 reduces the radiation exposure of the breast tissue of the patient 22 from about 20% to about 40%.

Rotation of components on gantry 12 and the operation of x-ray source 14 are governed by a control mechanism 26 of CT system 10. Control mechanism 26 includes an x-ray controller 28 that provides power and timing signals to x-ray source 14 and a gantry motor controller 30 that controls the rotational speed and position of components on gantry 12. A data acquisition system (DAS) 32 in control mechanism 26 samples analog data from detector elements 20 and converts the data to digital signals for subsequent processing. An image reconstructor 34 receives sampled and digitized x-ray data from DAS 32 and performs high-speed image reconstruction. The reconstructed image is applied as an input to a computer 36, which stores the image in a storage device 38. Image reconstructor 34 can be specialized hardware or computer programs executing on computer 36.

Computer 36 also receives commands and scanning parameters from an operator via console 40 that has a keyboard. An associated cathode ray tube display or other suitable type of display 42 allows the operator to observe the reconstructed image and other data from computer 36. The operator supplied commands and parameters are used by computer 36 to provide control signals and information to DAS 32, x-ray controller 28, and gantry motor controller 30. In addition, computer 36 operates a table motor controller 44, which controls a motorized table 46 to position patient 22 in gantry 12. Particularly, table 46 moves portions of patient 22 through gantry opening 48.

It is to be understood that an ECG machine 60 may monitor electrical signals indicative of the cardiac phase of patient 22. A signal from ECG machine 60 is used by computer 36 to prospectively gate the operation of imaging system 10 so that times for acquiring data for reconstructing images at particular portions of the cardiac phase of patient 22 are determined. Radiation source 14 may also be gated in accordance with the determined times to thereby reduce the radiation dose to which patient 22 is subjected and to ensure that data at selected kVps is acquired at particular times relative to the cardiac cycle.

Accordingly, the shield 100 may be used to reduce the radiation exposure of the breast tissue of the patient 22 during CT calcium scoring procedures without substantially reducing the accuracy of the calcium score.

Although a third generation CT system is described herein, it is to be understood that the radiation shield 100 and methods described herein equally apply to any generation of CT systems including, but not limited to, fourth generation CT systems (stationary detector—rotating x-ray source), fifth generation CT systems (stationary detector and x-ray source), and to electron beam computed tomographic (EBCT) imaging systems. Also, it is to be understood that a CT imaging system 10 that is used to perform the calcium scoring can be a dual-energy CT imaging system or more generally, a multiple energy CT imaging system having two or more x-ray radiation energies, and may be a dual-energy or multiple energy EBCT imaging system. Additionally, it is contemplated that the benefits of the invention accrue to imaging modalities other than CT.

### EXAMPLES

The following examples set forth in Tables 1-22 and Charts 1-22 were performed in accordance with the invention and are intended to illustrate the invention without, however, limiting it.

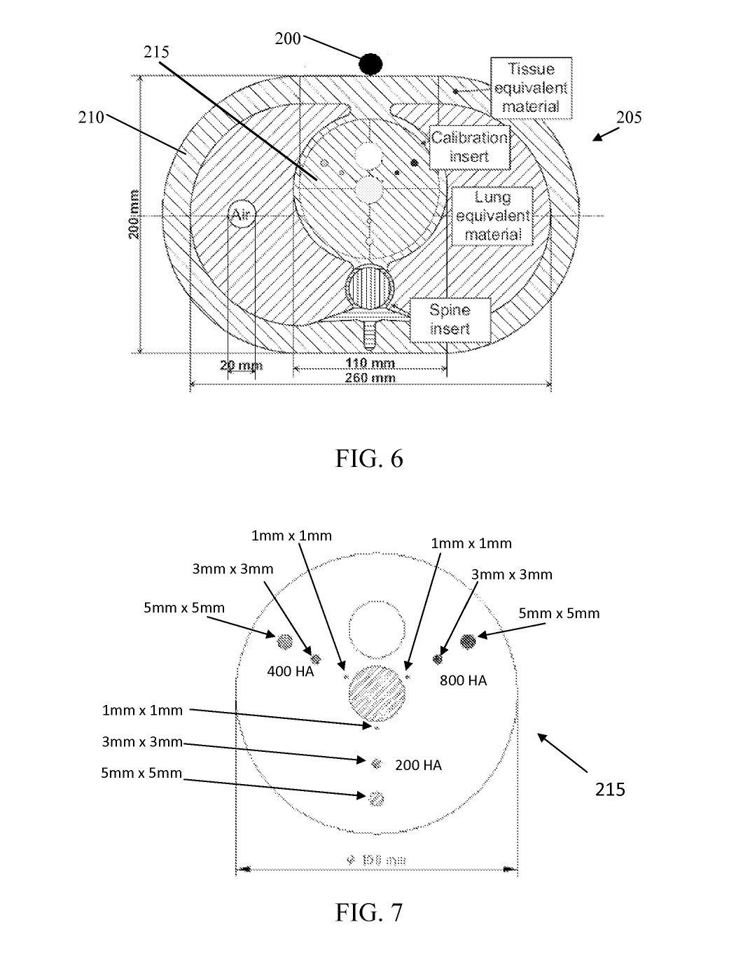

The effects of the shield 100 (over a range of different Pb equivalents) were studied using a 16 Row CT system manufactured by Siemens Medical Solutions USA, Inc., under the trademark SOMATOM® (also referred to herein as Siemens 16 Row: Siemens Somatom), a 64-slice CT system manufactured by Philips, under the trademark BRILLIANCE® (also referred to herein as Philips 64: Philips Brilliance or Philips 64 Row: Philips Brilliance), a 64-slice CT system manufactured by Siemens Medical Solutions USA, Inc., under the trademark SOMATOM SENSATION® (also referred to herein as Siemens 64 Row: Siemens Sensation), and a 64 Row Dual Source CT system manufactured by Siemens Medical Solutions USA, Inc., under the trademark SOMATOM DEFINITION [trade] (also referred to herein as Siemens 64 Row: Siemens Dual Tube). CT scans were performed on each system according to standard calcium score protocols and dose measurements were obtained using an ion chamber 200 (Victoreen Model 660-D x-ray and CT Exposure Measurement Instrument) positioned on an Anthropomorphic Cardio CT Phantom 205 (manufactured by QRM GmbH of Germany) (hereinafter referred to as “the phantom 205”), as shown in FIG. 6.

As best shown in FIGS. 6 and 7, the phantom 205 includes two parts: an anthropomorphic thorax phantom body 210 and a cardiac calcification insert 215. The body 210 includes artificial lung lobes, a spine insert, and shell of soft tissue equivalent material. The plastics used in the phantom 205 mimic human tissues in the thorax with respect to density and attenuation characteristics. By positioning the ion chamber 200 on the surface of the phantom body 210 as shown in FIG. 6, the radiation exposure that a human breast tissue is exposed to during a CT scan for calcium scoring on each system was determined. Ten CT scans for each system were performed according to standard calcium score protocol and the radiation dosage was measured by the ion chamber 200 as set forth in Tables 1, 2, 9 and 16 (under “0 Shield” or “Shield 0”).

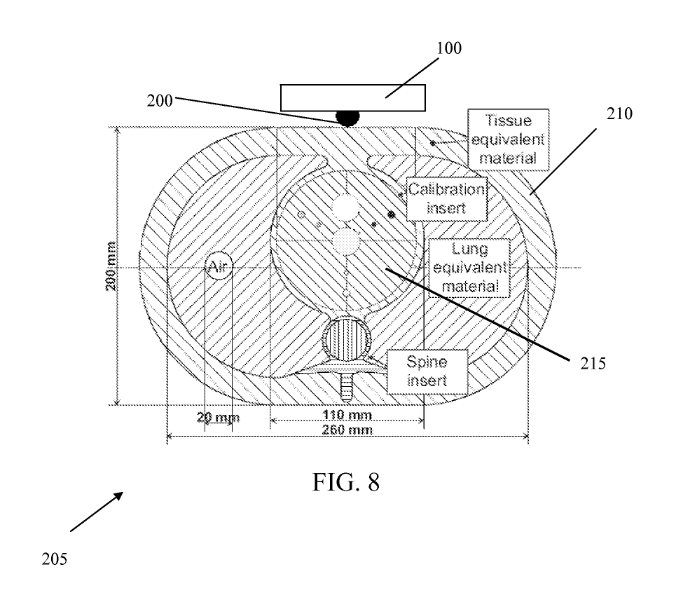

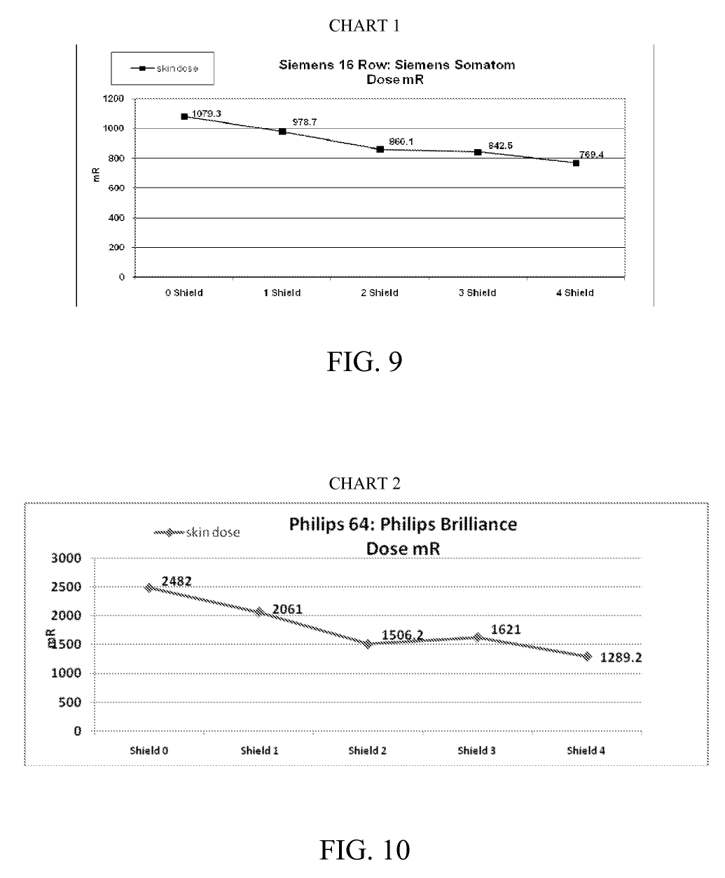

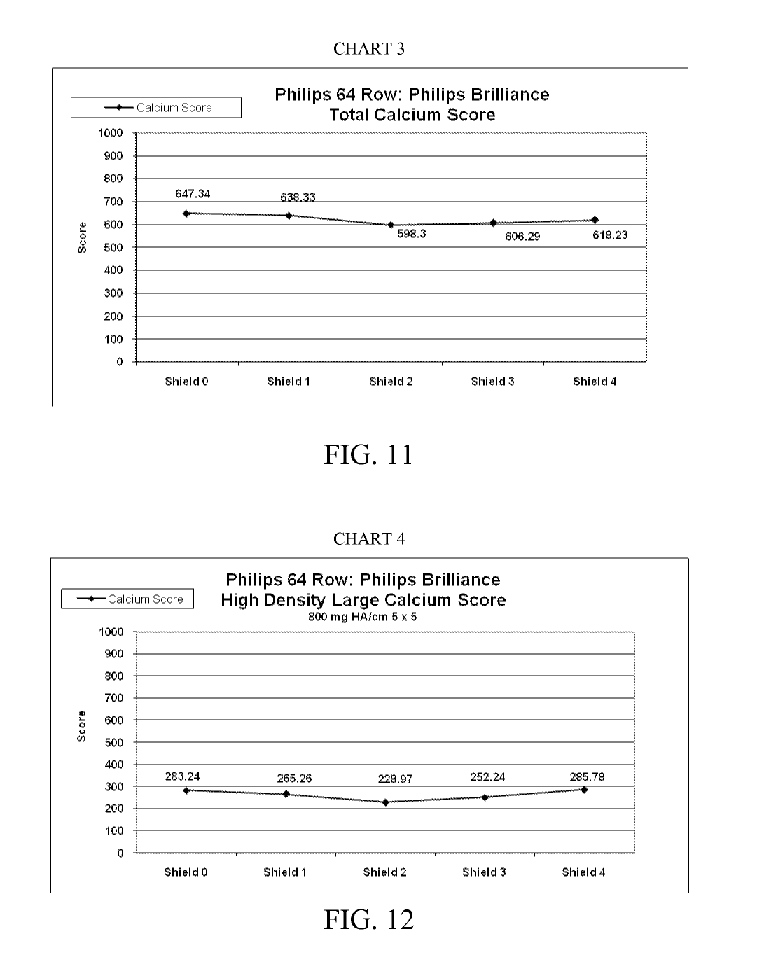

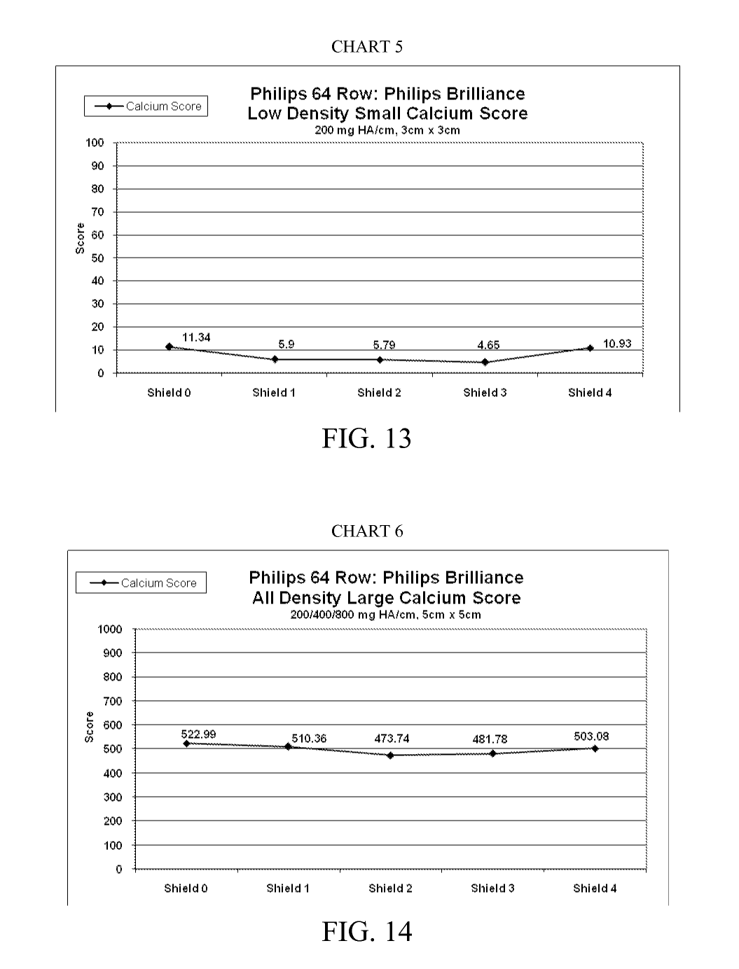

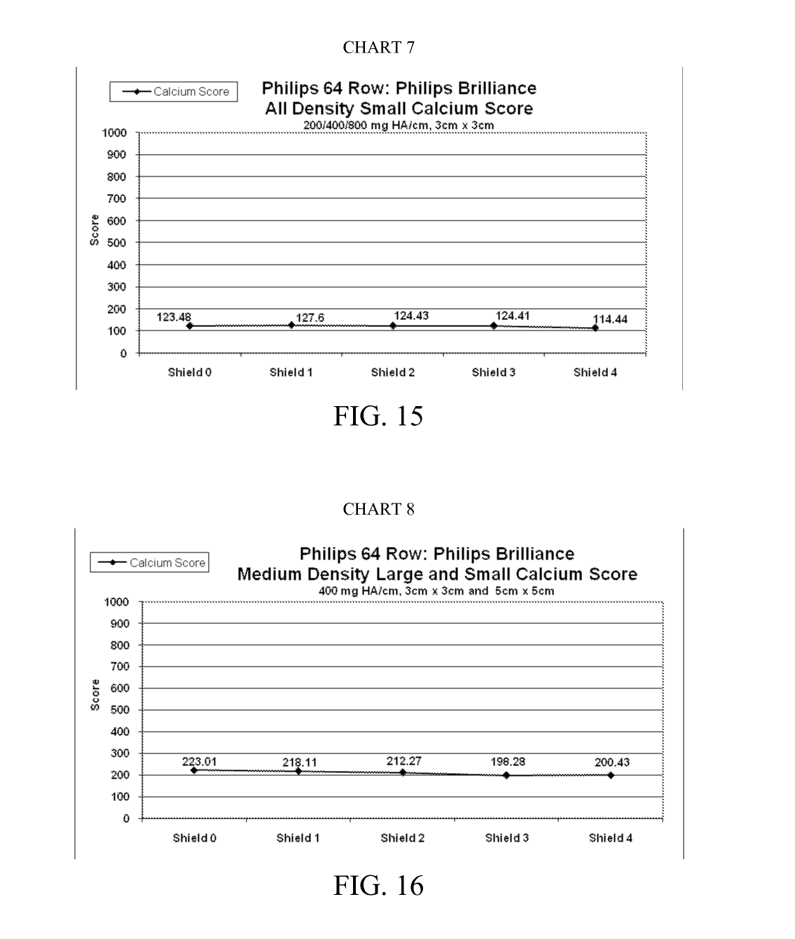

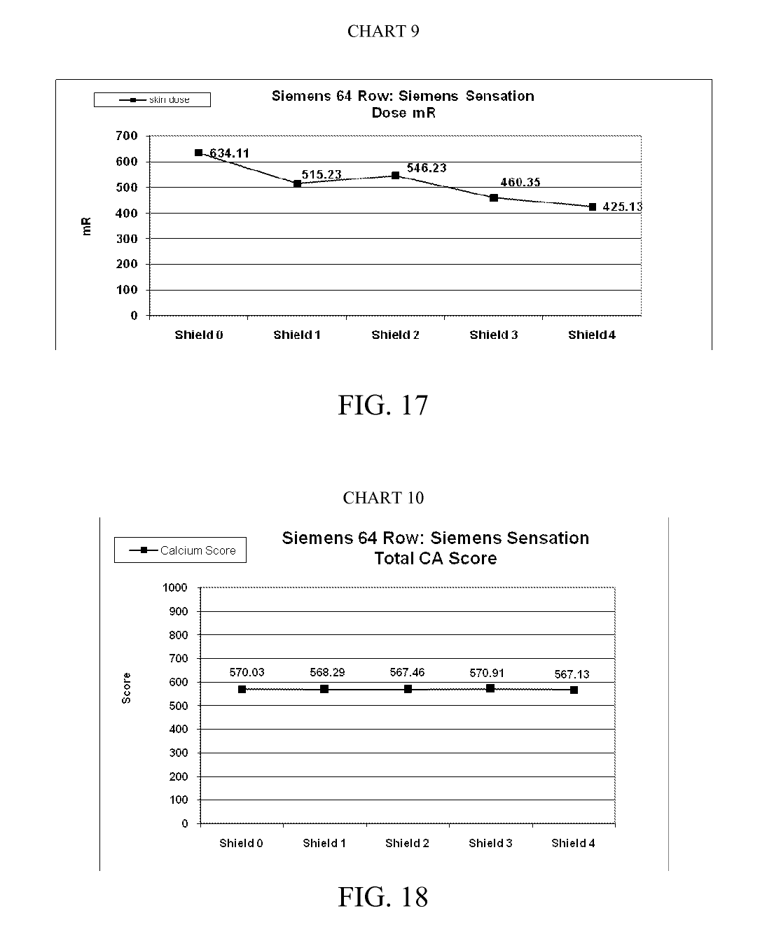

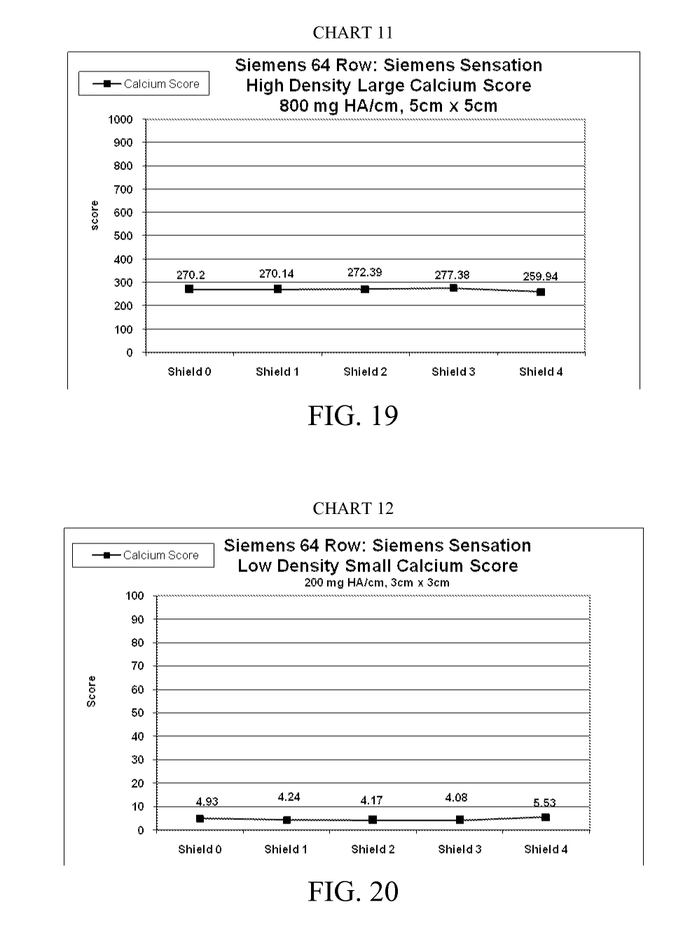

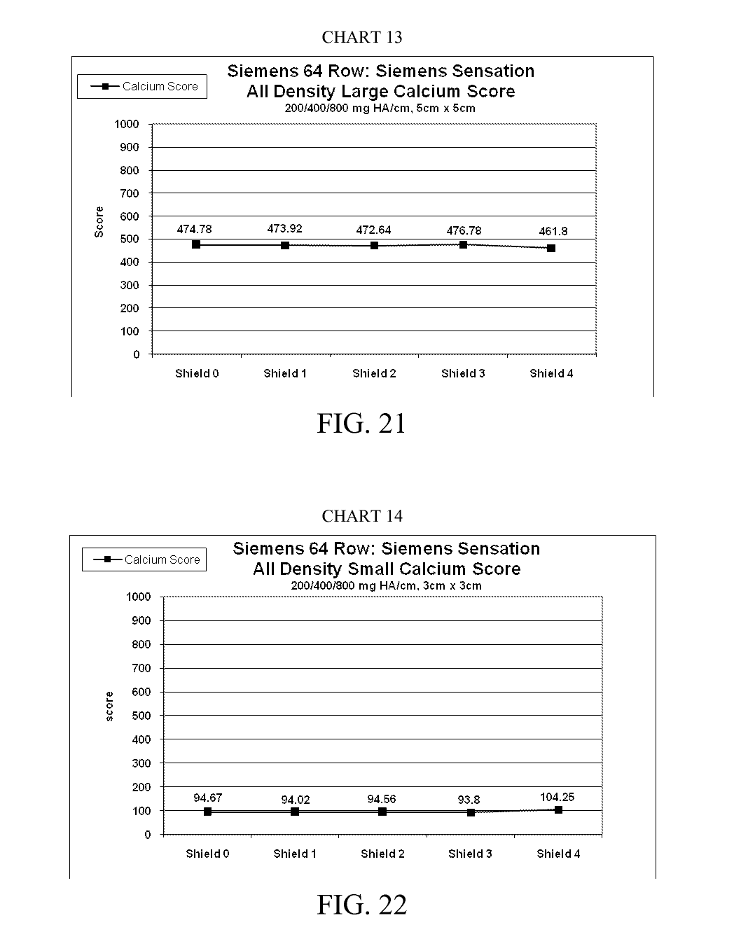

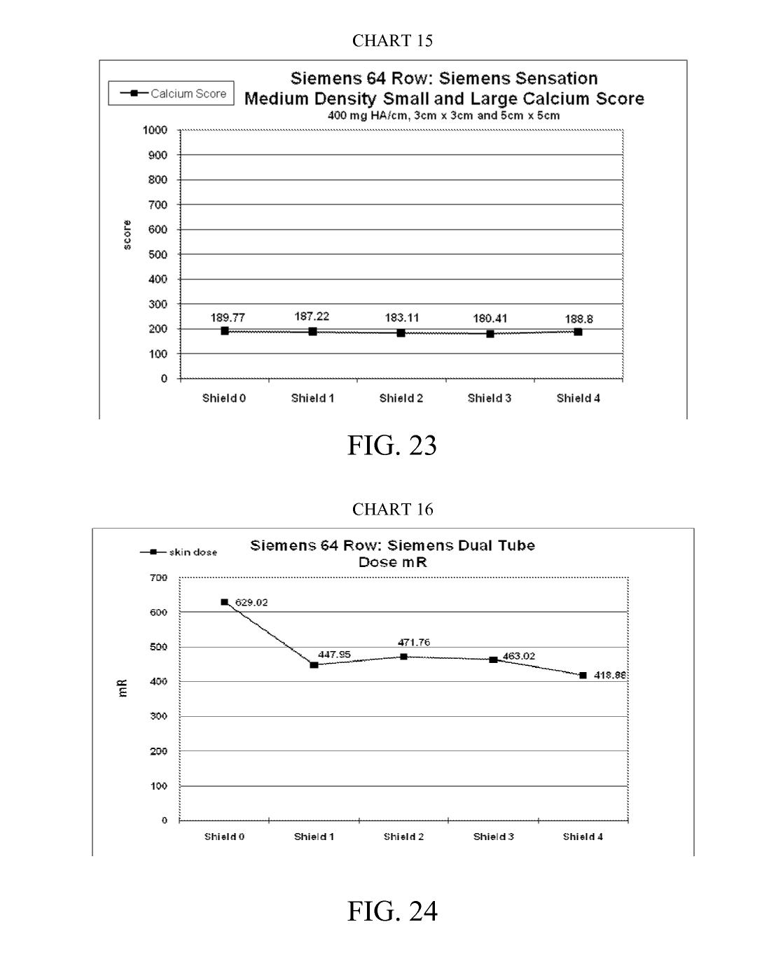

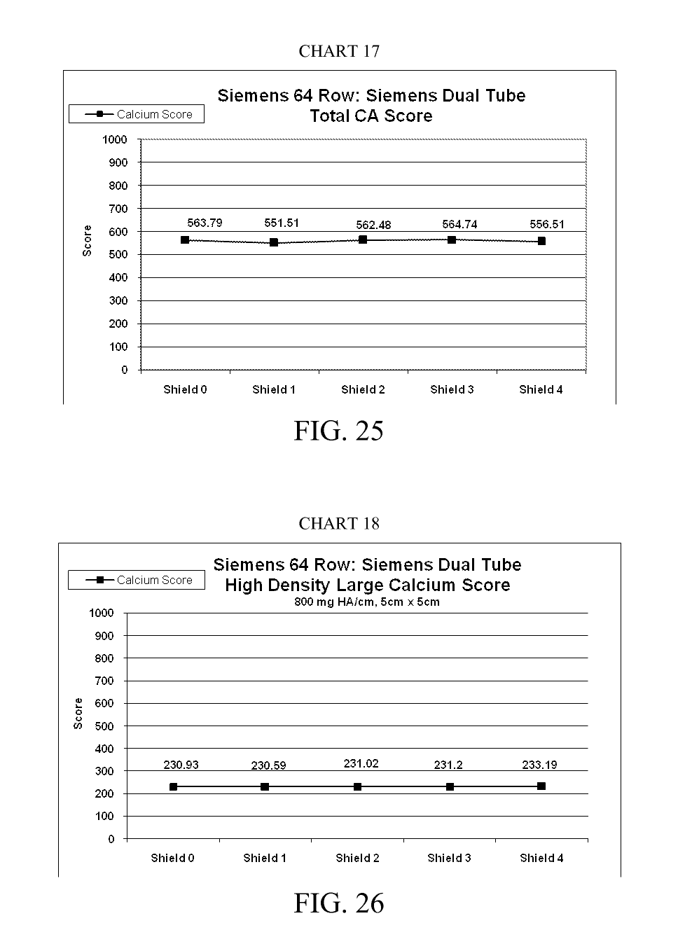

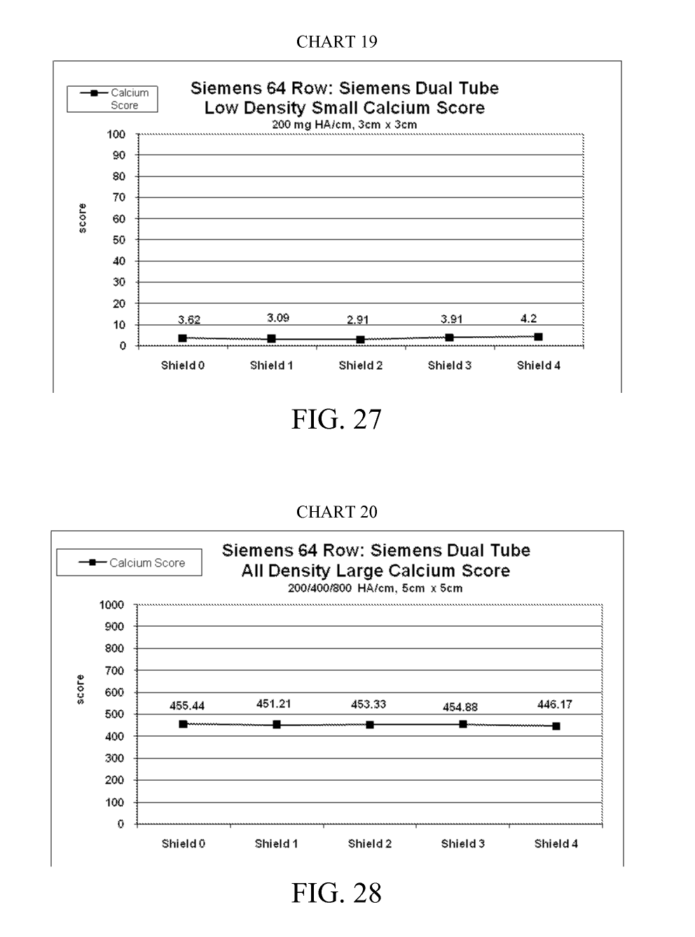

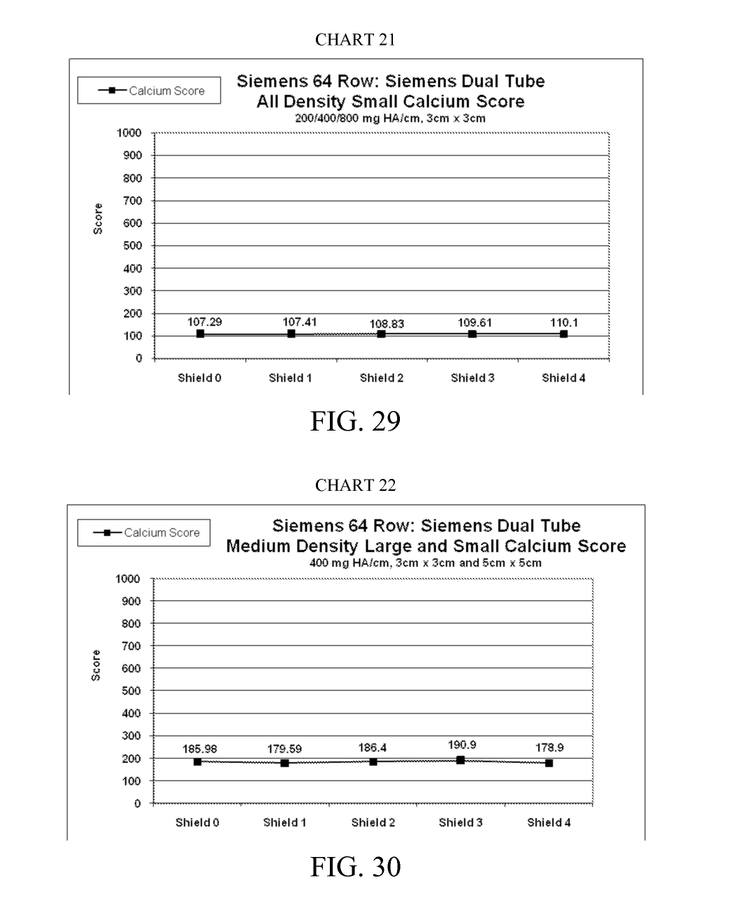

To measure the amount the shield 100 reduced the radiation dosage to the breast tissue of a human during a CT scan for calcium scoring, the shield 100 was positioned over the ion chamber 200 as shown in FIG. 8. Ten CT scans were performed on each system according to standard calcium score protocols for shields 100 having one layer of radioabsorbent material with a 0.060 mm Pb equivalent (hereinafter referred to as “layer” or “layers”), two layers (for a total of 0.120 mm Pb equivalent), three layers (for a total of 0.180 mm Pb equivalent) and four layers (for a total of 0.240 mm Pb equivalent). Each layer was an AttenuRad CT Breast Shield System (Female Pediatric) (from F&L Medical Products) comprised of a bismuth impregnated synthetic rubber and having a 0.060 mm Pb equivalent. The radiation dosages measured by the ion chamber 200 for each scan are set forth in Tables 1, 2, 9 and 16 (under “1 Shield” or “Shield 1 ” for one layer, “2 Shield” or “Shield 2 ” for two layers, “3 Shield” or “Shield 3 ” for three layers, and “4 Shield” or “Shield 4 ” for four layers).

So that the impact of the shield 100 (over a range of different Pb equivalents) could be evaluated on the total calcium score as well as different calcium densities and sizes, the cardiac calcification insert 215 contained nine cylindrical calcifications varying in size and hydroxyapatite (HA) density, as shown in Table A, embedded in a tissue-equivalent solid of about 35 HU (+/−5 HU).

TABLE A

| HA density | Length | Diameter | Volumn | Area | HA mass |
|---|---|---|---|---|---|
| [mg/cm³] | [mm] | [mm] | [mm³] | [mm²] | [mg] |
| 200 | 5.0 | 5.0 | 98.2 | 19.6 | 19.6 |
| 200 | 3.0 | 3.0 | 21.2 | 7.1 | 4.2 |
| 200 | 1.0 | 1.0 | 0.8 | 0.8 | 0.2 |
| 400 | 5.0 | 5.0 | 98.2 | 19.6 | 39.3 |
| 400 | 3.0 | 3.0 | 21.2 | 7.1 | 8.5 |
| 400 | 1.0 | 1.0 | 0.8 | 0.8 | 0.3 |
| 800 | 5.0 | 5.0 | 98.2 | 19.6 | 78.5 |
| 800 | 3.0 | 3.0 | 21.2 | 7.1 | 17.0 |
| 800 | 1.0 | 1.0 | 0.8 | 0.8 | 0.6 |

To establish baseline calcium scores, the ion chamber 200 was removed and ten CT scans for the Philips 64: Philips Brilliance, Siemens 64 Row: Siemens Sensation, and a Siemens 64 Row: Siemens Dual Tube systems were performed according to standard calcium score protocol without the shield 100 (“Shield 0 ” or “0 Shield”) for the total calcium score (shown in Tables 3, 10 and 17 ), high density large calcium scores (Tables 4, 11, 18), low density small calcium scores (Tables 5, 12, 19), all density large calcium scores (Tables 6, 13, 20), all density small calcium score (Tables 7, 14, 21) and medium density large and small calcium score (Tables 8, 15, 22). The image data sets were individually analyzed using market available coronary artery calcium scoring software.

Because of the concern for an increase in image noise, coronary calcium score was measured with the shields 100 having one layer (“Shield 1” or “1 Shield”), two layers (“Shield 2 ” or “2 Shield”), three layers (“Shield 3 ” or “3 Shield”) and four layers (“Shield 4 ” or “4 Shield”) of radioabsorbent material (as described above). Ten CT scans for the Philips 64: Philips Brilliance, Siemens 64 Row: Siemens Sensation, and a Siemens 64 Row: Siemens Dual Tube systems were performed according to standard calcium score protocol with the shield 100 having one layer having a 0.060 mm Pb equivalent, two layers having a total of 0.120 mm Pb equivalent, three layers having a total of 0.180 mm Pb equivalent and four layers having a total of 0.240 mm Pb equivalent for total calcium score (Tables 3, 10, 17), high density large calcium scores (Tables 4, 11, 18), low density small calcium scores (Tables 5, 12, 19), all density large calcium scores (Tables 6, 13, 20), all density small calcium score (Tables 7, 14, 21) and medium density large and small calcium score (Tables 8, 15, 22). The image data sets were individually analyzed using market available coronary artery calcium scoring software.

TABLE 1
Siemens 16 Row: Siemens Somatom

| | No Shield | Shield | Shield | Shield | Shield |
|---|---|---|---|---|---|
| | (0 Layer) | (1 Layer) | (2 Layer) | (3 Layer) | (4 Layer) |
| Trials | Doses (mr) | Doses (mr) | Doses (mr) | Doses (mr) | Doses (mr) |
| 1 | 691 | 1100 | 1040 | 1030 | 950* |
| 2 | 923 | 1320* | 786 | 805 | 870 |
| 3 | 707 | 540 | 1160 | 975 | 785 |
| 4 | 1390 | 525 | 1170* | 558 | 514 |
| 5 | 1490* | 1310 | 978 | 520 | 885 |
| 6 | 715 | 829 | 499 | 957 | 935 |
| 7 | 1090 | 975 | 737 | 1070* | 415 |
| 8 | 1440 | 728 | 1120 | 440 | 550 |
| 9 | 1490* | 1150 | 1170* | 1060 | 855 |
| 10 | 857 | 1310 | 540 | 1010 | 935 |
| STDV | 343.2 | 309.8 | 261.1 | 245.2 | 199.3 |
| AVE | 1079.3 | 978.7 | 860.1 | 842.5 | 769.4 |
| P-Val | 0 | 0.505 | 0.229 | 0.192 | 0.065 |

*Maximum Radiation Dosage

TABLE 2
Philips 64: Philips Brilliance

| | 0 Shield | 1 Shield | 2 Shield | 3 Shield | 4 Shield |
|---|---|---|---|---|---|
| Trials | Doses (mr) | Doses (mr) | Doses (mr) | Doses (mr) | Doses (mr) |
| 1 | 3080* | 1650 | 994 | 2020 | 1390 |
| 2 | 2980 | 1020 | 1100 | 1740 | 1400 |
| 3 | 2490 | 1970 | 2000 | 1220 | 885 |
| 4 | 1960 | 2600 | 1330 | 1730 | 957 |
| 5 | 3030 | 2680* | 2330* | 1310 | 1040 |
| 6 | 3030 | 2680* | 928 | 2060* | 1020 |
| 7 | 2340 | 2650 | 2300 | 1260 | 1520 |
| 8 | 1600 | 2680* | 960 | 960 | 1800* |
| 9 | 2350 | 1550 | 2120 | 1850 | 1390 |
| 10 | 1960 | 1130 | 1000 | 2060* | 1490 |
| STDV | 534.0 | 681.2 | 603.4 | 401.1 | 297.3 |
| AVE | 2482 | 2061 | 1506.2 | 1621 | 1289.2 |
| P-Val | 0 | 0.182 | 0.003 | 0.001 | 0 |

*Maximum Radiation Dosage

TABLE 3
Philips 64: Philips Brilliance
Total Calcium Score

| | Shield 0 | Shield 1 | Shield 2 | Shield 3 | Shield 4 |
|---|---|---|---|---|---|
| | Calcium | Calcium | Calcium | Calcium | Calcium |
| Trials | Score | Score | Score | Score | Score |
| 1 | 651.9 | 602.3 | 602.9 | 600.8 | 609.3 |
| 2 | 647.1 | 655.6 | 612.4 | 617.7 | 600.7 |
| 3 | 667.6 | 624.2 | 588.2 | 625.2 | 631.3 |
| 4 | 644.4 | 632.2 | 594.8 | 590.6 | 597.1 |
| 5 | 648.6 | 656.3 | 606.0 | 577.7 | 607.2 |
| 6 | 647.9 | 618.2 | 589.6 | 616.6 | 615.3 |
| 7 | 653.5 | 650.7 | 604.4 | 583.3 | 643.3 |
| 8 | 626.5 | 665.4 | 587.3 | 625.6 | 630.7 |
| 9 | 648.1 | 621.9 | 609.7 | 623.6 | 624.6 |
| 10 | 637.8 | 656.5 | 587.6 | 601.8 | 622.7 |
| STDV | 10.6 | 21.2 | 9.8 | 18.0 | 14.8 |
| AVE | 647.3 | 638.3 | 598.3 | 606.3 | 618.2 |
| P-Val | 0 | 0.3418 | 0 | 0.0002 | 0.0005 |

TABLE 4
Philips 64: Philips Brilliance
High Density Large Calcium Score (800 mg HA/cm, 5 cm × 5 cm)

| | Shield 0 | Shield 1 | Shield 2 | Shield 3 | Shield 4 |
|---|---|---|---|---|---|
| | Calcium | Calcium | Calcium | Calcium | Calcium |
| Trials | Score | Score | Score | Score | Score |
| 1 | 276.6 | 234.5 | 223.3 | 256.4 | 281.0 |
| 2 | 303.7 | 283.2 | 241.1 | 250.5 | 275.6 |
| 3 | 279.4 | 249.7 | 227.4 | 277.0 | 300.1 |
| 4 | 277.1 | 256.9 | 222.3 | 231.4 | 283.6 |
| 5 | 305.7 | 282.0 | 234.0 | 222.9 | 282.3 |
| 6 | 277.3 | 248.7 | 227.9 | 261.4 | 271.7 |
| 7 | 278.4 | 283.8 | 232.6 | 233.6 | 278.2 |
| 8 | 279.6 | 280.5 | 221.8 | 276.6 | 275.0 |
| 9 | 280.4 | 247.5 | 238.3 | 255.1 | 305.7 |
| 10 | 274.4 | 285.9 | 221.3 | 257.6 | 304.7 |
| STDV | 11.4 | 19.6 | 7.2 | 18.2 | 12.8 |
| AVE | 283.2 | 265.3 | 229.0 | 252.2 | 285.8 |
| P-Val | 0 | 0.0111 | 0 | 0.0037 | 0.6874 |

TABLE 5
Philips 64: Philips Brilliance
Low Density Small Calcium Score (200 mg HA/cm, 3 cm × 3 cm)

| | Shield 0 | Shield 1 | Shield 2 | Shield 3 | Shield 4 |
|---|---|---|---|---|---|
| | Calcium | Calcium | Calcium | Calcium | Calcium |
| Trials | Score | Score | Score | Score | Score |
| 1 | 10.94 | 4.83 | 6.48 | 4.45 | 11.19 |
| 2 | 11.19 | 4.96 | 6.61 | 4.96 | 11.70 |
| 3 | 10.94 | 9.41 | 5.09 | 4.96 | 10.94 |
| 4 | 10.43 | 3.94 | 5.98 | 3.56 | 10.94 |
| 5 | 11.44 | 5.09 | 3.81 | 3.94 | 10.94 |
| 6 | 12.21 | 5.98 | 5.72 | 4.45 | 10.68 |
| 7 | 11.95 | 4.20 | 6.23 | 4.96 | 11.44 |
| 8 | 11.44 | 10.05 | 6.23 | 5.09 | 10.68 |
| 9 | 11.44 | 5.47 | 5.98 | 5.72 | 10.94 |
| 10 | 11.44 | 5.09 | 5.85 | 4.45 | 9.92 |
| STDV | 0.51 | 2.10 | 0.82 | 0.62 | 0.48 |
| AVE | 11.34 | 5.90 | 5.79 | 4.65 | 10.93 |
| P-Val | 0.00 | 0.00 | 0.00 | 0.00 | 0.12 |

TABLE 6
Philips 64: Philips Brilliance
All Density Large Calcium Score
(200/400/800 mg HA/cm, 5 cm × 5 cm)

| | Shield 0 | Shield 1 | Shield 2 | Shield 3 | Shield 4 |
|---|---|---|---|---|---|
| | Calcium | Calcium | Calcium | Calcium | Calcium |
| Trials | Score | Score | Score | Score | Score |
| 1 | 521.7 | 474.0 | 472.3 | 478.9 | 488.0 |
| 2 | 543.6 | 529.6 | 481.7 | 493.2 | 497.4 |
| 3 | 527.8 | 495.8 | 471.5 | 501.6 | 516.0 |
| 4 | 524.9 | 505.1 | 467.7 | 464.5 | 484.1 |
| 5 | 520.1 | 526.7 | 479.1 | 453.3 | 483.8 |
| 6 | 525.5 | 491.1 | 471.2 | 489.4 | 500.7 |
| 7 | 531.3 | 524.7 | 474.7 | 460.2 | 524.5 |
| 8 | 504.8 | 531.5 | 470.2 | 504.4 | 519.2 |
| 9 | 524.8 | 496.7 | 486.3 | 496.4 | 509.6 |
| 10 | 505.5 | 528.6 | 462.9 | 475.8 | 507.4 |
| STDV | 11.4 | 20.4 | 6.9 | 18.0 | 14.7 |
| AVE | 523.0 | 510.4 | 473.7 | 481.8 | 503.1 |
| P-Val | 0.0000 | 0.1442 | 0.0000 | 0.0002 | 0.0111 |

TABLE 7
Philips 64: Philips Brilliance
All Density Small Calcium Score
(200/400/800 mg HA/cm, 3 cm × 3 cm)

| | Shield 0 | Shield 1 | Shield 2 | Shield 3 | Shield 4 |
|---|---|---|---|---|---|
| | Calcium | Calcium | Calcium | Calcium | Calcium |
| Trials | Score | Score | Score | Score | Score |
| 1 | 129.6 | 128.3 | 130.6 | 121.9 | 121.3 |
| 2 | 123.5 | 126.0 | 130.7 | 124.5 | 102.6 |
| 3 | 118.5 | 128.4 | 116.2 | 123.6 | 114.2 |
| 4 | 119.0 | 126.1 | 127.2 | 126.1 | 112.2 |
| 5 | 127.8 | 129.1 | 126.9 | 124.4 | 122.7 |
| 6 | 121.6 | 126.7 | 118.4 | 127.2 | 113.9 |
| 7 | 121.3 | 125.5 | 129.1 | 123.1 | 118.8 |
| 8 | 120.7 | 133.3 | 117.1 | 120.7 | 110.5 |
| 9 | 122.6 | 125.3 | 123.5 | 127.2 | 113.8 |
| 10 | 130.3 | 127.4 | 124.7 | 125.5 | 114.4 |
| STDV | 4.27 | 2.38 | 5.49 | 2.16 | 5.73 |
| AVE | 123.5 | 127.6 | 124.4 | 124.4 | 114.4 |
| P-Val | 0.0000 | 0.0237 | 0.5673 | 0.5652 | 0.0006 |

TABLE 8
Philips 64: Philips Brilliance
Medium Density Large and Small Calcium Score (400 mg HA/cm, 3 cm × 3 cm and 5 cm × 5 cm)

| | Shield 0 | Shield 1 | Shield 2 | Shield 3 | Shield 4 |
|---|---|---|---|---|---|
| | Calcium | Calcium | Calcium | Calcium | Calcium |
| Trials | Score | Score | Score | Score | Score |
| 1 | 230.2 | 213.5 | 223.8 | 190.0 | 196.8 |
| 2 | 219.0 | 216.7 | 211.6 | 214.0 | 193.5 |
| 3 | 230.2 | 221.1 | 205.2 | 194.0 | 184.9 |
| 4 | 233.3 | 221.1 | 216.8 | 202.1 | 183.7 |
| 5 | 194.3 | 217.6 | 214.1 | 199.3 | 195.7 |
| 6 | 229.6 | 214.3 | 207.3 | 198.4 | 222.4 |
| 7 | 236.1 | 212.1 | 211.3 | 194.9 | 229.5 |
| 8 | 198.8 | 224.7 | 208.5 | 190.4 | 221.4 |
| 9 | 229.6 | 224.1 | 213.6 | 213.8 | 187.2 |
| 10 | 229.1 | 216.0 | 210.4 | 186.2 | 189.2 |
| STDV | 14.7 | 4.4 | 5.3 | 9.5 | 17.2 |
| AVE | 223.0 | 218.1 | 212.3 | 198.3 | 200.4 |
| P-Val | 0.0000 | 0.3765 | 0.0507 | 0.0014 | 0.0155 |

TABLE 9
Siemens 64 Row: Siemens Sensation

| | Shield 0 Doses | Shield 1 Doses | Shield 2 Doses | Shield 3 Doses | Shield 4 Doses |
|---|---|---|---|---|---|
| Trials | (mr) | (mr) | (mr) | (mr) | (mr) |
| 1 | 785.3 | 474.4 | 551.62 | 479.35 | 521.7 |
| 2 | 432.8 | 456.8 | 465.71 | 325.43 | 560.2 |
| 3 | 801.4* | 702.5 | 564.88 | 522.51 | 318.7 |
| 4 | 770.7 | 643.2 | 318.05 | 607.76 | 306.0 |
| 5 | 392.9 | 729.3* | 643.97 | 349.19 | 562.0* |
| 6 | 749.1 | 524.3 | 550.43 | 304.55 | 490.4 |
| 7 | 637.9 | 362.3 | 477.21 | 608.67* | 313.9 |
| 8 | 597.4 | 446.1 | 651.51* | 331.42 | 558.2 |
| 9 | 799.8 | 402.0 | 626.72 | 568.80 | 297.3 |
| 10 | 373.9 | 411.6 | 619.02 | 505.88 | 322.8 |
| STDV | 175.7 | 130.9 | 103.3 | 121.7 | 121.5 |
| AVE | 634.1 | 515.2 | 546.9 | 460.4 | 425.1 |
| P-Val | 0 | 0.1098 | 0.2544 | 0.0092 | 0.026 |

* = Maximum Radiation Dosage

TABLE 10
Siemens 64 Row: Siemens Sensation
Total Calcium Score

| | Shield 0 | Shield 1 | Shield 2 | Shield 3 | Shield 4 |
|---|---|---|---|---|---|
| | Calcium | Calcium | Calcium | Calcium | Calcium |
| Trials | Score | Score | Score | Score | Score |
| 1 | 572.6 | 569.7 | 573.4 | 576.8 | 548.2 |
| 2 | 565.9 | 564.8 | 578.4 | 585.5 | 577.3 |
| 3 | 566.2 | 572.2 | 564.8 | 560.4 | 557.2 |
| 4 | 572.0 | 580.7 | 562.0 | 572.2 | 573.3 |
| 5 | 572.2 | 542.9 | 575.6 | 585.1 | 570.3 |
| 6 | 571.9 | 569.9 | 573.8 | 579.6 | 571.3 |
| 7 | 550.3 | 570.1 | 548.8 | 569.7 | 566.9 |
| 8 | 587.7 | 581.6 | 572.0 | 569.4 | 554.7 |
| 9 | 575.6 | 576.8 | 565.9 | 571.0 | 574.6 |
| 10 | 565.9 | 554.2 | 559.9 | 539.4 | 577.5 |
| STDV | 9.4 | 11.9 | 9.0 | 13.5 | 10.2 |
| AVE | 570.03 | 568.29 | 567.46 | 570.91 | 567.13 |
| P-Val | 0 | 0.6821 | 0.3405 | 0.8591 | 0.5743 |

TABLE 11
Siemens 64 Row: Siemens Sensation
High Density Large Calcium Score (800 mg HA/cm, 5 cm × 5 cm)

| | Shield 0 | Shield 1 | Shield 2 | Shield 3 | Shield 4 |
|---|---|---|---|---|---|
| | Calcium | Calcium | Calcium | Calcium | Calcium |
| Trials | Score | Score | Score | Score | Score |
| 1 | 286.4 | 258.5 | 287.7 | 288.3 | 260.6 |
| 2 | 285.5 | 254.3 | 283.2 | 289.0 | 286.7 |
| 3 | 279.8 | 281.1 | 259.2 | 284.3 | 261.0 |
| 4 | 260.6 | 283.9 | 251.8 | 285.0 | 263.4 |
| 5 | 255.0 | 256.0 | 287.2 | 284.4 | 281.6 |
| 6 | 282.7 | 281.8 | 286.9 | 288.0 | 263.4 |
| 7 | 256.4 | 254.6 | 261.3 | 285.3 | 260.2 |
| 8 | 259.9 | 288.1 | 283.5 | 259.2 | 235.6 |
| 9 | 279.0 | 279.3 | 264.9 | 252.5 | 255.0 |
| 10 | 256.7 | 263.8 | 258.2 | 257.8 | 231.9 |
| STDV | 13.4 | 13.8 | 14.5 | 14.6 | 17.0 |
| AVE | 270.20 | 270.14 | 272.39 | 277.38 | 259.94 |
| P-Val | 0 | 0.9921 | 0.6749 | 0.2101 | 0.1017 |

TABLE 12
Siemens 64 Row: Siemens Sensation
Low Density Small Calcium Score (200 mg HA/cm, 3 cm × 3 cm)

| | Shield 0 | Shield 1 | Shield 2 | Shield 3 | Shield 4 |
|---|---|---|---|---|---|
| | Calcium | Calcium | Calcium | Calcium | Calcium |
| Trials | Score | Score | Score | Score | Score |
| 1 | 4.6 | 4.8 | 3.4 | 3.5 | 6.3 |
| 2 | 3.7 | 3.7 | 5.1 | 4.8 | 3.0 |
| 3 | 4.2 | 4.4 | 4.1 | 3.2 | 2.6 |
| 4 | 8.8 | 4.1 | 4.4 | 3.7 | 6.7 |
| 5 | 4.6 | 4.6 | 3.9 | 4.2 | 2.8 |
| 6 | 3.5 | 4.4 | 4.2 | 5.3 | 3.0 |
| 7 | 8.1 | 4.4 | 3.0 | 3.9 | 4.2 |
| 8 | 4.2 | 3.7 | 5.1 | 3.9 | 4.7 |
| 9 | 3.7 | 3.5 | 3.9 | 4.2 | 11.0 |
| 10 | 3.9 | 4.8 | 4.6 | 4.1 | 11.0 |
| STDV | 1.9 | 0.5 | 0.7 | 0.6 | 3.2 |
| AVE | 4.93 | 4.24 | 4.17 | 4.08 | 5.53 |
| P-Val | 0 | 0.284 | 0.3121 | 0.2532 | 0.6288 |

TABLE 13
Siemens 64 Row: Siemens Sensation
All Density Large Calcium Score (200/400/800 mg HA/cm, 5 cm × 5 cm)

| | Shield 0 | Shield 1 | Shield 2 | Shield 3 | Shield 4 |
|---|---|---|---|---|---|
| | Calcium | Calcium | Calcium | Calcium | Calcium |
| Trials | Score | Score | Score | Score | Score |
| 1 | 476.8 | 472.4 | 476.8 | 479.3 | 449.7 |
| 2 | 478.6 | 468.9 | 483.2 | 494.4 | 479.3 |
| 3 | 471.5 | 473.6 | 471.7 | 474.3 | 458.1 |
| 4 | 475.8 | 490.6 | 475.0 | 474.9 | 472.9 |
| 5 | 469.4 | 446.5 | 480.0 | 488.8 | 471.9 |
| 6 | 473.8 | 475.6 | 478.7 | 479.2 | 478.7 |
| 7 | 472.4 | 483.5 | 455.3 | 473.6 | 465.5 |
| 8 | 453.0 | 481.0 | 474.2 | 474.4 | 447.4 |
| 9 | 493.2 | 491.6 | 469.2 | 475.0 | 445.9 |
| 10 | 483.3 | 455.5 | 462.3 | 453.9 | 448.6 |
| STDV | 10.3 | 14.4 | 8.5 | 10.6 | 13.4 |
| AVE | 474.78 | 473.92 | 472.64 | 476.78 | 461.80 |
| P-Val | 0 | 0.875 | 0.65 | 0.7007 | 0.0446 |

TABLE 14
Siemens 64 Row: Siemens Sensation
All Density Small Calcium Score
(200/400/800 mg HA/cm, 3 cm × 3 cm)

| | Shield 0 | Shield 1 | Shield 2 | Shield 3 | Shield 4 |
|---|---|---|---|---|---|
| | Calcium | Calcium | Calcium | Calcium | Calcium |
| Trials | Score | Score | Score | Score | Score |
| 1 | 88.0 | 97.2 | 96.6 | 97.5 | 98.2 |
| 2 | 93.8 | 95.6 | 95.2 | 90.3 | 97.7 |
| 3 | 94.3 | 98.6 | 93.1 | 85.2 | 98.4 |
| 4 | 96.3 | 90.1 | 86.4 | 97.3 | 98.9 |
| 5 | 93.8 | 96.5 | 95.2 | 95.6 | 97.7 |
| 6 | 98.0 | 93.8 | 95.0 | 101.9 | 91.9 |
| 7 | 99.5 | 97.9 | 93.5 | 95.4 | 95.4 |
| 8 | 96.8 | 85.7 | 97.5 | 94.7 | 107.3 |
| 9 | 94.3 | 99.8 | 95.6 | 94.9 | 128.5 |
| 10 | 91.9 | 85.0 | 97.5 | 85.2 | 128.5 |
| STDV | 3.3 | 5.3 | 3.2 | 5.4 | 13.3 |
| AVE | 94.67 | 94.02 | 94.56 | 93.80 | 104.25 |
| P-Val | 0 | 0.7556 | 0.9494 | 0.6235 | 0.0675 |

TABLE 15
Siemens 64 Row: Siemens Sensation
Medium Density Large and Small Calcium Score
(400 mg HA/cm, 3 cm × 3 cm and 5 cm × 5 cm)

| | Shield 0 | Shield 1 | Shield 2 | Shield 3 | Shield 4 |
|---|---|---|---|---|---|
| | Calcium | Calcium | Calcium | Calcium | Calcium |
| Trials | Score | Score | Score | Score | Score |
| 1 | 168.4 | 200.8 | 180.9 | 179.2 | 177.4 |
| 2 | 180.6 | 203.8 | 173.5 | 171.0 | 182.3 |
| 3 | 177.0 | 180.6 | 200.8 | 168.8 | 179.9 |
| 4 | 203.3 | 176.3 | 192.6 | 177.7 | 181.3 |
| 5 | 203.3 | 179.9 | 180.2 | 176.7 | 184.4 |
| 6 | 177.4 | 180.9 | 179.2 | 177.4 | 206.5 |
| 7 | 202.1 | 194.1 | 179.9 | 177.2 | 179.9 |
| 8 | 180.9 | 182.3 | 179.1 | 205.6 | 196.1 |
| 9 | 203.5 | 193.6 | 178.3 | 200.3 | 195.9 |
| 10 | 201.2 | 179.9 | 186.6 | 170.2 | 204.3 |
| STDV | 14.0 | 9.9 | 8.1 | 12.5 | 10.9 |
| AVE | 189.77 | 187.22 | 183.11 | 180.41 | 188.80 |
| P-Val | 0 | 0.6898 | 0.2225 | 0.1373 | 0.8598 |

TABLE 16
Siemens 64 Row: Siemens Dual Tube

| | 0 Shield Doses | 1 Shield Doses | 2 Shield Doses | 3 Shield Doses | 4 Shield Doses |
|---|---|---|---|---|---|
| Trials | (mr) | (mr) | (mr) | (mr) | (mr) |
| 1 | 727.8 | 256.34 | 300.5 | 562.6 | 530.3 |
| 2 | 600.4 | 435.82 | 632.8* | 578.0* | 299.3 |
| 3 | 328.2 | 561.93 | 538.6 | 239.4 | 520.6 |
| 4 | 703.4 | 285.42 | 587.0 | 510.5 | 529.2 |
| 5 | 501.6 | 278.83 | 466.0 | 440.4 | 453.7 |
| 6 | 782.3 | 713.97* | 551.9 | 570.6 | 490.8 |
| 7 | 468.3 | 510.22 | 247.3 | 449.4 | 236.2 |
| 8 | 806.9* | 403.32 | 575.0 | 530.8 | 261.6 |
| 9 | 781.9 | 680.74 | 565.5 | 460.3 | 332.3 |
| 10 | 589.4 | 352.90 | 253.0 | 288.2 | 534.8* |
| ST DV | 159.4 | 148.0 | 116.8 | 116.8 | 122.3 |
| AVE | 629.0 | 448.0 | 471.8 | 463.0 | 418.9 |
| P-Val | 0 | 0.0225 | 0.0346 | 0.0161 | 0.0039 |

* = Maximum Radiation Dosage

TABLE 17
Siemens 64 Row: Siemens Dual Tube
Total Calcium Score

| | Shield 0 | Shield 1 | Shield 2 | Shield 3 | Shield 4 |
|---|---|---|---|---|---|
| | Calcium | Calcium | Calcium | Calcium | Calcium |
| Trials | Score | Score | Score | Score | Score |
| 1 | 551.3 | 554.6 | 561.2 | 576.9 | 556.5 |
| 2 | 564.2 | 541.4 | 585.3 | 549.8 | 554.4 |
| 3 | 555.3 | 551.5 | 558.2 | 554.3 | 559.2 |
| 4 | 577.0 | 544.3 | 587.1 | 557.1 | 540.0 |
| 5 | 569.0 | 575.5 | 551.6 | 573.8 | 568.0 |
| 6 | 544.8 | 538.8 | 575.1 | 549.0 | 579.0 |
| 7 | 559.8 | 539.0 | 547.8 | 580.2 | 544.8 |
| 8 | 570.3 | 543.6 | 560.5 | 563.3 | 550.9 |
| 9 | 565.0 | 545.3 | 550.2 | 572.6 | 546.9 |
| 10 | 581.2 | 581.1 | 547.8 | 570.4 | 565.4 |
| STDV | 11.3 | 15.0 | 14.9 | 11.6 | 11.8 |
| AVE | 563.79 | 551.51 | 562.48 | 564.74 | 556.51 |
| P-Val | 0 | 0.0204 | 0.8367 | 0.8421 | 0.2617 |

TABLE 18
Siemens 64 Row: Siemens Dual Tube
High Density Large Calcium Score (800 mg HA/cm, 5 cm × 5 cm)

| | Shield 0 | Shield 1 | Shield 2 | Shield 3 | Shield 4 |
|---|---|---|---|---|---|
| | Calcium | Calcium | Calcium | Calcium | Calcium |
| Trials | Score | Score | Score | Score | Score |
| 1 | 238.5 | 231.7 | 230.2 | 229.6 | 232.8 |
| 2 | 234.4 | 232.3 | 228.2 | 228.0 | 236.2 |
| 3 | 231.2 | 232.3 | 236.1 | 232.3 | 236.8 |
| 4 | 230.7 | 229.1 | 231.2 | 234.0 | 230.8 |
| 5 | 225.9 | 229.1 | 230.7 | 236.5 | 231.6 |
| 6 | 232.3 | 231.6 | 230.2 | 234.9 | 234.4 |
| 7 | 229.4 | 232.8 | 237.7 | 229.1 | 232.4 |
| 8 | 227.0 | 226.5 | 227.3 | 230.2 | 230.7 |
| 9 | 228.6 | 230.3 | 231.6 | 227.5 | 233.3 |
| 10 | 231.3 | 230.2 | 227.0 | 229.9 | 232.9 |
| STDV | 3.7 | 2.0 | 3.5 | 3.1 | 2.1 |
| AVE | 230.93 | 230.59 | 231.02 | 231.20 | 233.19 |
| P-Val | 0 | 0.7265 | 0.9586 | 0.8783 | 0.0609 |

TABLE 19
Siemens 64 Row: Siemens Dual Tube
Low Density Small Calcium Score (200 mg HA/cm, 3 cm × 3 cm)

| | Shield 0 | Shield 1 | Shield 2 | Shield 3 | Shield 4 |
|---|---|---|---|---|---|
| | Calcium | Calcium | Calcium | Calcium | Calcium |
| Trials | Score | Score | Score | Score | Score |
| 1 | 2.0 | 2.8 | 3.3 | 4.1 | 4.6 |
| 2 | 3.0 | 1.5 | 2.8 | 3.0 | 3.3 |
| 3 | 3.7 | 4.5 | 1.5 | 2.1 | 3.2 |
| 4 | 4.5 | 4.0 | 3.7 | 3.8 | 5.9 |
| 5 | 4.2 | 2.2 | 2.6 | 4.1 | 1.5 |
| 6 | 3.6 | 2.5 | 2.5 | 4.6 | 3.6 |
| 7 | 4.0 | 2.9 | 3.8 | 5.0 | 3.3 |
| 8 | 2.9 | 2.8 | 2.1 | 2.8 | 4.1 |
| 9 | 3.8 | 1.5 | 3.6 | 4.1 | 4.4 |
| 10 | 4.5 | 6.2 | 3.2 | 5.5 | 8.1 |
| STDV | 0.8 | 1.4 | 0.7 | 1.0 | 1.8 |
| AVE | 3.62 | 3.09 | 2.91 | 3.91 | 4.20 |
| P-Val | 0 | 0.2346 | 0.0447 | 0.4007 | 0.3275 |

TABLE 20
Siemens 64 Row: Siemens Dual Tube
All Density Large Calcium Score
(200/400/800 mg HA/cm, 5 cm × 5 cm)

| | Shield 0 | Shield 1 | Shield 2 | Shield 3 | Shield 4 |
|---|---|---|---|---|---|
| | Calcium | Calcium | Calcium | Calcium | Calcium |
| Trials | Score | Score | Score | Score | Score |
| 1 | 446.7 | 444.9 | 459.8 | 466.7 | 445.5 |
| 2 | 456.9 | 436.2 | 470.1 | 439.5 | 445.4 |
| 3 | 447.1 | 441.3 | 444.9 | 448.8 | 443.2 |
| 4 | 467.4 | 437.4 | 467.1 | 446.2 | 428.0 |
| 5 | 462.0 | 464.9 | 444.2 | 464.5 | 456.2 |
| 6 | 440.1 | 483.4 | 465.3 | 445.0 | 472.9 |
| 7 | 456.4 | 460.1 | 443.8 | 466.1 | 438.4 |
| 8 | 456.2 | 436.8 | 455.3 | 453.5 | 442.8 |
| 9 | 457.3 | 438.9 | 437.8 | 465.6 | 439.5 |
| 10 | 464.3 | 468.2 | 445.0 | 452.9 | 449.8 |
| STDV | 8.5 | 16.7 | 11.6 | 10.1 | 11.9 |
| AVE | 455.44 | 451.21 | 453.33 | 454.88 | 446.17 |
| P-Val | 0 | 0.532 | 0.6775 | 0.8933 | 0.1418 |

TABLE 21
Siemens 64 Row: Siemens Dual Tube
All Density Small Calcium Score
(200/400/800 mg HA/cm, 3 cm × 3 cm)

| | Shield 0 | Shield 1 | Shield 2 | Shield 3 | Shield 4 |
|---|---|---|---|---|---|
| | Calcium | Calcium | Calcium | Calcium | Calcium |
| Trials | Score | Score | Score | Score | Score |
| 1 | 104.3 | 109.3 | 101.1 | 109.8 | 110.6 |
| 2 | 107.3 | 105.2 | 115.2 | 109.5 | 108.4 |
| 3 | 107.6 | 110.2 | 112.8 | 105.4 | 116.0 |
| 4 | 109.1 | 106.5 | 120.1 | 110.9 | 111.6 |
| 5 | 107.7 | 110.6 | 107.4 | 109.3 | 111.8 |
| 6 | 104.5 | 100.0 | 109.1 | 104.0 | 105.1 |
| 7 | 103.5 | 106.9 | 103.2 | 113.8 | 106.4 |
| 8 | 105.1 | 106.2 | 104.4 | 109.5 | 108.1 |
| 9 | 107.7 | 106.4 | 112.2 | 106.4 | 107.4 |
| 10 | 116.1 | 112.8 | 102.8 | 117.5 | 115.6 |
| STDV | 3.6 | 3.6 | 6.2 | 4.0 | 3.7 |
| AVE | 107.29 | 107.41 | 108.83 | 109.61 | 110.10 |
| P-Val | 0 | 0.9104 | 0.4919 | 0.0767 | 0.0126 |

TABLE 22
Siemens 64 Row: Siemens Dual Tube
Medium Density Large and Small Calcium Score
(400 mg HA/cm, 3 cm × 3 cm and 5 cm × 5 cm)

| | Shield 0 | Shield 1 | Shield 2 | Shield 3 | Shield 4 |
|---|---|---|---|---|---|
| | Calcium | Calcium | Calcium | Calcium | Calcium |
| Trials | Score | Score | Score | Score | Score |
| 1 | 168.8 | 174.2 | 191.1 | 201.5 | 179.4 |
| 2 | 188.5 | 171.0 | 205.2 | 175.5 | 170.1 |
| 3 | 173.5 | 169.7 | 172.1 | 175.3 | 174.1 |
| 4 | 198.2 | 171.6 | 211.8 | 178.8 | 176.6 |
| 5 | 194.8 | 197.7 | 178.8 | 191.6 | 197.0 |
| 6 | 170.6 | 166.6 | 202.4 | 168.9 | 196.5 |
| 7 | 191.4 | 192.4 | 167.5 | 201.5 | 174.5 |
| 8 | 196.5 | 175.8 | 193.1 | 201.1 | 177.6 |
| 9 | 186.3 | 175.1 | 167.7 | 200.9 | 164.4 |
| 10 | 191.2 | 201.8 | 174.3 | 213.9 | 178.8 |
| STDV | 11.0 | 12.7 | 16.5 | 15.2 | 10.4 |
| AVE | 185.98 | 179.59 | 186.40 | 190.90 | 178.90 |
| P-Val | 0 | 0.1322 | 0.9473 | 0.3499 | 0.2009 |

For each CT scanner, as the Pb equivalency of the shield 100 was increased the radiation exposure detected by the ion chamber 200 under the shield 100 decreased (as shown in Tables 1, 2, 9 and 16). As seen in Tables 2-8, 10-15, and 17-22, the use of the shield 100 during the CT scan, regardless of the lead equivalency, did not substantially reduce the accuracy of any of the calcium scores.

For example, as shown in Table 1 for the Siemens 16 Row: Siemens Somatom, the average radiation exposure for the ten trials was 1079 mr without the shield 100, 978.7 mr with a shield 100 having one layer, 860.1 mr with a shield 100 having two layers, 842.5 mr with a shield 100 having three layers, and 769.4 mr with a shield 100 having four layers.

Further, as shown in Table 2 for the Philips 64: Philips Brilliance, the average radiation exposure for the ten trials was 2482 millirads (“mr”) without the shield 100, 2061 mr with a shield 100 having one layer, 1506.2 mr with a shield 100 having two layers, 1621 mr with a shield 100 having three layers, and 1289.2 mr with a shield 100 having four layers. As shown in Table 3, the accuracy of the total calcium score was not substantially reduced by the shield 100 (having any Pb equivalent). As shown in Table 3, the average total calcium score without the shield 100 was 647.3, 638.3 with the shield 100 having one layer, 598.3 with the shield 100 having two layers, 606.3 with the shield 100 having three layers, and 618.2 with the shield having four layers. As extensive evidence of CAD is defined as a score of >400, the small change in the total calcium score due to use of the shield 100 (for all layers) as shown in Table 3 would not change medical management. As further shown in Tables 4 through 8, use of the shield 100 described herein during calcium scoring does not reduce the accuracy of the calcium scoring for any density or size.

As shown in Table 9 for the Siemens 64 Row: Siemens Sensation, the average radiation exposure for the ten trials was 634.1 mr without the shield 100, 515.2 mr with a shield 100 having one layer, 546.9 mr with a shield 100 having two layers, 460.4 mr with a shield 100 having three layers, and 425.1 mr with a shield 100 having four layers. As shown in Table 10, the total calcium score was not negatively impacted by the shield 100 as the average total calcium score without the shield 100 was 570.03, 568.29 with the shield 100 having one layer, 567.46 with the shield 100 having two layers, 570.91 with the shield 100 having three layers, and 567.13 with the shield having four layers. As extensive evidence of CAD is defined as a score of >400, the small change in the total calcium score due to use of the shield 100 (for all layers) as shown in Table 10 would not change medical management. As shown in Tables 11 through 15, use of the radiation shield 100 described herein during calcium scoring did not reduce the accuracy of the calcium scoring for any density or size.

As shown in Table 16 for the Siemens 64 Row: Siemens Dual Tube, the average radiation for the ten trials was 629 mr without the shield 100, 448 mr with a shield 100 having one layer, 471.8 mr with a shield 100 having two layers, 463 mr with a shield 100 having three layers, and 418.9 mr with a shield 100 having four layers. As shown in Table 17, the total calcium score was not negatively impacted by the shield 100 as the average total calcium score without the shield 100 was 563.79, 551.51 with the shield 100 having one layer, 562.48 with the shield 100 having two layers, 564.74 with the shield 100 having three layers, and 556.51 with the shield having four layers. As extensive evidence of CAD is defined as a score of >400, the small change in the total calcium score due to use of the shield 100 (for all layers) as shown in Table 17 would not change medical management. As shown in Tables 18 through 22, use of the shield 100 described herein during calcium scoring did not reduce the accuracy of the calcium scoring for any density or size.

Accordingly, the shield 100 described herein reduces radiation dosage of the underlying tissue during CT scans for calcium scoring without substantially reducing the accuracy of the calcium score or the following medical management of the patient.

Although this device has been shown and described with respect to a certain embodiment or embodiments, it will be apparent to those skilled in the art upon reading of this specification and the annexed drawings that many alternatives, modifications and variations may be made. In addition, while a particular feature may have been described above with respect to only one or more several illustrated embodiments, such feature may be combined with one or more other features of the other embodiments, as may be desired or advantageous for any given or particular application. Accordingly, the present invention is intended to embrace all such alternatives, modifications, variations and combinations.

I claim:

1. A method of generating a diagnostic output for a target area located adjacent a radiosensitive secondary area using a computed tomography (CT) device, the CT device having an x-ray source and an x-ray detector, the method comprising:
transmitting x-rays from the x-ray source through a radio-absorbent shield, the target area and the radiosensitive secondary area to the x-ray detector;
receiving x-rays from the x-ray source at the x-ray detector;
generating signals based on the received x-rays; and
processing signals generated by the x-ray detector to provide a diagnostic output related to the target area.

2. The method of claim 1, further comprising:
positioning a patient between the x-ray source and the x-ray detector to scan a patient cross section including the target area and the secondary area;
placing the radio-absorbent shield over the radiosensitive secondary area and over the primary area.

3. The method of claim 1, wherein the target area includes coronary arteries.

4. The method of claim 3, wherein the radiosensitive secondary area includes breast tissue.

5. The method of claim 1, wherein the processing includes generating a calcium score representative of coronary artery calcification in the target area.

6. A method of generating a calcium score corresponding to a target area of a patient, wherein the target area is adjacent to a radiosensitive secondary area, the method comprising:
transmitting x-rays from an x-ray source through a shield positioned over the target area and the secondary area, through the target area and the radiosensitive secondary area to at least one x-ray detector, wherein the shield is radio-absorbent;
receiving x-rays from the x-ray source at the at least one x-ray detector;
generating signals based on the received x-rays; and
processing signals generated by the at least one x-ray detector to generate a calcium score corresponding to the target area.

7. The method of claim 6, further comprising:
positioning a patient between the x-ray source and the x-ray detector to scan a patient cross section including the target area and the radiosensitive secondary area;
placing the shield over the radiosensitive secondary area and over the primary area.

8. The method of claim 6, wherein the target area includes coronary arteries.

9. The method of claim 8, wherein the radiosensitive secondary area includes breast tissue.

10. The method of claim 7, wherein the shield comprises:
a plurality of layers of radio-absorbent material;
a cover at least partially surrounding the plurality of layers of radio-absorbent material;
wherein the shield is configured for placement over the target area and over the radiosensitive secondary area adjacent to the target area; and
wherein the plurality of layers of radio-absorbent material have a Pb equivalent of up to about 0.240 mm Pb equivalent.

11. The method of claim 10, wherein the plurality of layers of radio-absorbent material have a Pb equivalent from about 0.060 mm Pb equivalent to about 0.240 mm Pb equivalent.

12. The method of claim 10, wherein the shield includes four layers of radio-absorbent material, with each of the four layers of radio-absorbent material having a Pb equivalent of about 0.060 mm Pb equivalent.

13. The method of claim 10, wherein the shield is configured for placement over a cardiac target area and the radiosensitive secondary area includes breast tissue adjacent to the cardiac area.

14. A shield for use in connection with computed tomography (CT) calcium scoring of a target area of a patient, the shield comprising:
a plurality of layers of radio-absorbent material;
a cover at least partially surrounding the plurality of layers of radio-absorbent material;
wherein the shield is configured for placement over the target area and over a radiosensitive area adjacent to the target area; and
wherein the plurality of layers of radio-absorbent material have a Pb equivalent of up to about 0.240 mm Pb equivalent.

15. The shield of claim 14, wherein the plurality of layers of radio-absorbent material have a Pb equivalent from about 0.060 mm Pb equivalent to about 0.240 mm Pb equivalent.

16. The shield of claim 14, wherein the shield includes four layers of radio-absorbent material, with each of the four layers of radio-absorbent material having a Pb equivalent of about 0.060 mm Pb equivalent.

17. The shield of claim 14, wherein the shield is configured for placement over a cardiac target area and the radiosensitive area includes breast tissue adjacent to the cardiac area.

18. A method for detecting coronary artery calcification using a computed tomography (CT) system having an x-ray source and at least one x-ray detector, the method comprising:
positioning a patient between the x-ray source and the at least one x-ray detector to scan a patient cross section including a target area and a radiosensitive secondary area;
placing a shield partially transparent to x-rays over the radiosensitive secondary area and over the primary area;
transmitting x-rays from the x-ray source through the shield, the target area and the radiosensitive secondary area to the at least one x-ray detector;
receiving x-rays from the x-ray source at the x-ray detector;
generating signals based on the received x-rays; and
processing signals generated by the at least one x-ray detector to generate a calcium score corresponding to the target area.

19. The method of claim 18, wherein the shield comprises:
a plurality of layers of radio-absorbent material;
a cover at least partially surrounding the plurality of layers of radio-absorbent material;
wherein the shield is configured for placement over the target area and over the radiosensitive secondary area adjacent to the target area; and
wherein the plurality of layers of radio-absorbent material have a Pb equivalent of up to about 0.240 mm Pb equivalent.

20. The method of claim 18, wherein the plurality of layers of radio-absorbent material have a Pb equivalent from about 0.060 mm Pb equivalent to about 0.240 mm Pb equivalent.

21. The method of claim 18, wherein the shield includes four layers of radio-absorbent material, with each of the four layers of radio-absorbent material having a Pb equivalent of about 0.060 mm Pb equivalent.

22. A shield for reducing the x-ray dose of a radiosensitive area of a patient during computed tomography (CT) calcium scoring of the patient, the shield comprising:
a body positionable over the radiosensitive area; and
a radio-absorbent material secured to the body that attenuates greater than 56% of the x-rays passing through the radio-absorbent material to the radiosensitive area without substantially affecting the accuracy of the calcium score.

23. The shield of claim 22, wherein the radio-absorbent material attenuates about 58% or more of the x-rays passing through the material to the radiosensitive area.

24. The shield of claim 22, wherein the radio-absorbent material attenuates about 60% or more of the x-rays passing through the material to the radiosensitive area.

25. The shield of claim 22, wherein the radio-absorbent material has a Pb equivalent of about 0.215 mm Pb or greater.

26. The shield of claim 22, wherein the radio-absorbent material has a Pb equivalent of about 0.22 mm Pb or greater.

27. The shield of claim 22, wherein the radio-absorbent material has a Pb equivalent of about 0.24 mm Pb or greater.

28. The shield of claim 22, wherein the x-rays attenuated by the radio-absorbent material are in the range of about 100 kV to about 120 kV.

29. The shield of claim 22, wherein the body is a polymer and the radio-absorbent material is impregnated therein.

30. The shield of claim 29, wherein the body is latex and the radio-absorbent material is bismuth.
